SEQUENCE LISTING (29 sequences)

SEQ ID NO: 1 - protein, 716 aa, Rhodotorula glutinis
MAPSLDSISHSFANGVASAKQAVNGASTNLAVAGSHLPTTQVTQVDIVEKMLAAPTDSTLELDGYSLNLGDVVSAARKGRPVRVKDSDEIRSKIDKSVEFLRSQLSMSVYGVTTGFGGSADTRTEDAISLQKALLEHQLCGVLPSSFDSFRLGRGLENSLPLEVVRGAMTIRVNSLTRGHSAVRLVVLEALTNFLNHGITPIVPLRGTISASGDLSPLSYIAAAISGHPDSKVHVVHEGKEKILYAREAMALFNLEPVVLGPKEGLGLVNGTAVSASMATLALHDAHMLSLLSQSLTAMTVEAMVGHAGSFHPFLHDVTRPHPTQIEVAGNIRKLLEGSRFAVHHEEEVKVKDDEGILRQDRYPLRTSPQWLGPLVSDLIHAHAVLTIEAGQSTTDNPLIDVENKTSHHGGNFQAAAVANTMEKTRLGLAQIGKLNFTQLTEMLNAGMNRGLPSCLAAEDPSLSYHCKGLDIAAAAYTSELGHLANPVTTHVQPAEMANQAVNSLALISARRTTESNDVLSLLLATHLYCVLQAIDLRAIEFEFKKQFGPAIVSLIDQHFGSAMTGSNLRDELVEKVNKTLAKRLEQTNSYDLVPRWHDAFSFAAGTVVEVLSSTSLSLAAVNAWKVAAAESAISLTRQVRETFWSAASTSSPALSYLSPRTQILYAFVREELGVKARRGDVFLGKQEVTIGSNVSKIYEAIKSGRINNVLLKMLA

SEQ ID NO: 2 - protein, 737 aa, Phanerochaete chrysosporium
MPSRIDYYTSSGNGYAQSRKSSAIYPASASTGHAAPSTERKPELLDKFVEAYDELQSYREGKPVIVDGHNLSIPAVAATARFGAAVVLDENPETHERVLQSRRVIVDKVSTQRSVYGVSTGFGGSADTRTSDPLQLGHALLQHQHVGVLPTQTESPLPALPLGDPLATTSMPEAWVRGAILIRMNSLIRGHSGVRWELIEKMGELLRENITPLVPLRGSISASGDLSPLSYIAGTLIGSPAIRVFDGPASYGARRILPSNIALANHGVAPIPLSSKEHLGILNGTAFSASVGALALNEAVHLSLLAQVCTAMGTEAMIGAVGSFDAFIHDTARPHPGQVEVARNVRTLLEDSQMAVKAEDEVHIAEDEGELRQDRYPLRTAAQFLGPQIEDILSAHETVTLECNSTTDNPLIDGETGTVHHGGNFQAMAVTNAMEKTRLAIHHIGKLLFAQATELINPMMNRGLPPNLAATDPSHNYFAKGVDIHLAAYVGELGFLASPVSSHIQSAEMHNQAVNSLALVSARYTISALDVLSLLTAAYLYVLCQALDLRAMHNDLQSSLSAIVRELLPKHFPSAAKRADALLPILERTIFRALNSSSSADCKARMVSVAASTTTPLVDFLSADAALASELANITAFRTELATRAADALTTLRTQYLEGARGAAPASKYLGKTRPVYEFVRVTLNVPMHGRENLHNFEMGPGVEDGIIGNNISTIYEAIRDGKMQNVVMQLVKSIKA

SEQ ID NO: 3 - protein, 689 aa, Trichosporon cutaneum
MFIETNVAKPASTKAMNAGSAKAAPVEPFATYAHSQATKTVSIDGHTMKVGDVVAVARHGAKVELAASVAGPVRASVDFKESKKHTSIYGVTTGFGGSADTRTSDTEALQISLLEHQLCGFLPTDATYEGMLLAAMPIPIVRGAMAVRVNSCVRGHSGVRLEVLQSFADFINRGLVPCVPLRGTISASGDLSPLSYIAGAICGHPDVKVFDTAASPPTVLTSPEAIAKYGLKTVKLASKEGLGLVNGTAVSAAAGALALYDAECLAIMSQTNTVLTVEALDGHVGSFAPFIQEIRPHAGQIEAARNIRHMLGGSKLAVHEESELLADQDAGILRQDRYALRTSAQWIGPQLEALGLARQQIETELNSTTDNPLIDVEGGMFHHGGNFQAMAVTSAMDSARIVLQNLGKLSFAQVTELINCEMNHGLPSNLAGSEPSTNYHCKGLDIHCGAYCAELGFLANPMSNHVQSTEMHNQSVNSMAFASARRTMEANEVLSLLLGSQMYCATQALDLRVMEVKFKMAIVKLLNETLTKHFAAFLTPEQLAKLNTHAAITLYKRLNQTPSWDSAPRFEDAAKHLVGVIMDALMVNDDITDLTNLPKWKKEFAKEAGNLYRSILVATTADGRNDLEPAEYLGQTRAVYEAVRSELGVKVRRGDVAEGKSGKSIGSSVAKIVEAMRDGRLMGAVGKMF

SEQ ID NO: 4 - protein, 526 aa, Rhodobacter sphaeroides
VKPMLAMSPPKPAVELDRHIDLDQAHAVASGGARIVLAPPARDRCRASEARLGAVIREARHVYGLTTGFGPLANRLISGENVRTLQANLVHHLASGVGPVLDWTTARAMVLARLVSIAQGASGASEGTIARLIDLLNSELAPAVPSRGTVGASGDLTPLAHMVLCLQGRGDFLDRDGTRLDGAEGLRRGRLQPLDLSHRDALALVNGTSAMTGIALVNAHACRHLGNWAVALTALLAECLRGRTEAWAAALSDLRPHPGQKDAAARLRARVDGSARVVRHVIAERRLDAGDIGTEPEAGQDAYSLRCAPQVLGAGFDTLAWHDRVLTIELNAVTDNPVFPPDGSVPALHGGNFMGQHVALTSDALATAVTVLAGLAERQIARLTDERLNRGLPPFLHRGPAGLNSGFMGAQVTATALLAEMRATGPASIHSISTNAANQDVVSLGTIAARLCREKIDRWAEILAILALCLAQAAELRCGSGLDGVSPAGKKLVQALREQFPPLETDRPLGQEIAALATHLLQQSPV

SEQ ID NO: 5 - protein, 724 aa, Ustilago maydis
MAPTADVLPPVEASTRPGLLVQPSDTKLRKASSFRTEQVVIDGYNLKIQGLVASARYGHVTRPRPSAETRKRIDDSVQSLIAKLDGGESIYGINTGFGGSADSRTANTRALQLALLQMQQCGVLPVPSTFPTGEPSSAPFALPLTDTESSLIMPEAWVRGAIVVRLSSLMRGHSGVRWEVLDKMQKLFLQNNVTPVVPVRSSISASGDLSPLSYVAGALAGQRGIYCFVTDGRGQRVKVTADEACRMHKITPVQYEPKEALGLLNGTAFSASVAGLATYEAENLASLTQLTTAMAVEALKGTDASFAPFIHEIARPHPGQIKSAKFIRAHLSGSRLAEHLENEKHVLFSEDNGTLRQDRYTLQTASQWVGPGLEDIENAKRSVDFEINSTTDNPMIDPYDGDGRIHHGGNFQAMAMTNAVEKIRLALCAMGKMTFQQMTELVNPAMNRGLPANLASTPDLSLNFHAKGINIALASVTSELMFLGNPVSTHVQSAEMANQAFNSLALISGRQTLQAIECLSMIQAWSLYLLCQALDIRALQYKVAEQLPTLILASLHSHFGEWMDETKQQEIAAQVLKSMSKRLDETSSKDLRDRLVETYQDASSVLVRYFSELPSGGGADPLRNIVKWRATGVADTEKIYRQVTIEFLDNPYACHASHLLGKTKRAYEFVRKTLGVPMHGKENLNEFKGEFEQWNTTGGYVSVIYASIRDGELYNMLSELERDL

SEQ ID NO: 6 - protein, 716 aa, Petroselinum crispum
MENGNGATTNGHVNGNGMDFCMKTEDPLYWGIAAEAMTGSHLDEVKKMVAEYRKPVVKLGGETLTISQVAAISARDGSGVTVELSEAARAGVKASSDWVMDSMNKGTDSYGVTTGFGATSHRRTKQGGALQKELIRFLNAGIFGNGSDNTLPHSATRAAMLVRINTLLQGYSGIRFEILEAITKFLNQNITPCLPLRGTITASGDLVPLSYIAGLLTGRPNSKAVGPTGVILSPEEAFKLAGVEGGFFELQPKEGLALVNGTAVGSGMASMVLFEANILAVLAEVMSAIFAEVMQGKPEFTDHLTHKLKHHPGQIEAAAIMEHILDGSAYVKAAQKLHEMDPLQKPKQDRYALRTSPQWLGPQIEVIRSSTKMIEREINSVNDNPLIDVSRNKAIHGGNFQGTPIGVSMDNTRLAIAAIGKLMFAQFSELVNDFYNNGLPSNLSGGRNPSLDYGFKGAEIAMASYCSELQFLANPVTNHVQSAEQHNQDVNSLGLISSRKTSEAVEILKLMSTTFLVGLCQAIDLRHLEENLKSTVKNTVSSVAKRVLTMGVNGELHPSRFCEKDLLRVVDREYIFAYIDDPCSATYPLMQKLRQTLVEHALKNGDNERNLSTSIFQKIATFEDELKALLPKEVESARAALESGNPAIPNRIEECRSYPLYKFVRKELGTEYLTGEKVTSPGEEFEKVFIAMSKGEIIDPLLECLESWNGAPLPIC

SEQ ID NO: 7 - protein, 716 aa, Rhodosporidium toruloides mutant
MAPSLDSISHSFANGVASAKQAVNGASTNLAVAGSHLPTTQVTQVDIVEKMLAAPTDSTLELDGYSLNLGDVVSAARKGRPVRVKDSDEIRSKIDKSVEFLRSQLSMSVYGVTTGFGGSADTRTEDAISLQKALLEHQLCGVLPSSFDSFRLGRGLENSLPLEVVRGAMTIRVNSLTRGHSAVRLVVLEALTNFLNHGITPIVPLRGTISASGDLSPLSYIAAAISGHPDSKVHVVHEGKEKILYAREAMALFNLEPVVLGPKEGLGLVNGTAVSASMATLALHDAHMLSLLSQSLTAMTVEAMVGHAGSFHPFLHDVTRPHPTQIEVAGNIRKLLEGSRFAVHHEEEVKVKDDEGILRQDRYPLRTSPQWLGPLVSDLIHAHAVLTIEAGQSTTDNPLIDVENKTSHHGGNFQAAAVANTMEKTRLGLAQIGKLNFTQLTEMLNAGMNRGLPSCLAAEDPSLSYHCKGLDIAAAAYTSELGHLANPVTTHVQPAEMANQAVNSLALISARRTTESNDVLSLLLATHLYCVLQAIDLRATEFEFKKQFGPAIVSLIDQHFGSAMTGSNLRDELVEKVNKTLAKRLEQTNSYDLVPRWHDAFSFAAGTVVEVLSSTSLSLAAVNAWKVAAAESAISLTRQVRETFWSAASTSSPALSYLSPRTQILYAFVREELGVKARRGDVFLGKQEVTIGSNVSKIYEAIKSGRINNVLLKMLA

SEQ ID NO: 8 - protein, 716 aa, Rhodotorula glutinis mutant
  misc_feature (126)..(126): Xaa can be any naturally occurring amino acid
MAPSLDSISHSFANGVASAKQAVNGASTNLAVAGSHLPTTQVTQVDIVEKMLAAPTDSTLELDGYSLNLGDVVSAARKGRPVRVKDSDEIRSKIDKSVEFLRSQLSMSVYGVTTGFGGSADTRTEXAISLQKALLEHXLCGVLPSSFDSFRLGRGLENSLPLEVVRGAMTIRVNSLTRGHSAVRLVVLEALTNFLNHGITPIVPLRGTISASGDLSPLSYIAAAISGHPDSKVHVVHEGKEKILYAREAMALFNLEPVVLGPKEGLGLVNGTAVSASMATLALHDAHMLSLLSQSLTAMTVEAMVGHAGSFHPFLHDVTRPHPTQIEVAGNIRKLLEGSRFAVHHEEEVKVKDDEGILRQDRYPLRTSPQWLGPLVSDLIHAHAVLTIEAGQSTTDNPLIDVENKTSHHGGNFQAAAVANTMEKTRLGLAQIGKLNFTQLTEMLNAGMNRGLPSCLAAEDPSLSYHCKGLDIAAAAYTSELGHLANPVTTHVQPAEMANQAVNSLALISARRTTESNDVLSLLLATHLYCVLQAIDLRAXEFEFKKQFGPAIVSLIDQHFGSAMTGSNLRDELVEKVNKTLAKRLEQTNSYDLVPRWHDAFSFAAGTVVEVLSSTSLSLAAVNAWKVAAAESAISLTRQVRETFWSAASTSSPALSYLSPRTQILYAFVREELGVKARRGDVFLGKQEVTIGSNVSKIYEAIKSGRINNVLLKMLA

SEQ ID NO: 9 - protein, 716 aa, Rhodotorula glutinis mutant
  misc_feature (198)..(198): Xaa can be any naturally occurring amino acid
MAPSLDSISHSFANGVASAKQAVNGASTNLAVAGSHLPTTQVTQVDIVEKMLAAPTDSTLELDGYSLNLGDVVSAARKGRPVRVKDSDEIRSKIDKSVEFLRSQLSMSVYGVTTGFGGSADTRTEDAISLQKALLEHQLCGVLPSSFDSFRLGRGLENSLPLEVVRGAMTIRVNSLTRGHSAVRLVVLEALTNFLNHXITPIVPLRGTISASGDLSPLSYIAAAISGHPDSKVHVVHEGKEKILYAREAMALFNLEPVVLGPKEGLGLVNGTAVSASMATLALHDAHMLSLLSQSLTAMTVEAMVGHAGSFHPFLHDVTRPHPTQIEVAGNIRKLLEGSRFAVHHEEEVKVKDDEGILRQDRYPLRTSPQWLGPLVSDLIHAHAVLTIEAGQSTTDNPLIDVENKTSHHGGNFQAAAVANTMEKTRLGLAQIGKLNFTQLTEMLNAGMNRGLPSCLAAEDPSLSYHCKGLDIAAAAYTSELGHLANPVTTHVQPAEMANQAVNSLALISARRTTESNDVLSLLLATHLYCVLQAIDLRAXEFEFKKQFGPAIVSLIDQHFGSAMTGSNLRDELVEKVNKTLAKRLEQTNSYDLVPRWHDAFSFAAGTVVEVLSSTSLSLAAVNAWKVAAAESAISLTRQVRETFWSAASTSSPALSYLSPRTQILYAFVREELGVKARRGDVFLGKQEVTIGSNVSKIYEAIKSGRINNVLLKMLA

SEQ ID NO: 10 - protein, 716 aa, Rhodotorula glutinis mutant
  misc_feature (181)..(181): Xaa can be any naturally occurring amino acid
MAPSLDSISHSFANGVASAKQAVNGASTNLAVAGSHLPTTQVTQVDIVEKMLAAPTDSTLELDGYSLNLGDVVSAARKGRPVRVKDSDEIRSKIDKSVEFLRSQLSMSVYGVTTGFGGSADTRTEDAISLQKALLEHQLCGVLPSSFDSFRLGRGLENSLPLEVVRGAMTIRVNSLTRGHXAVRLVVLEALTNFLNHGITPIVPLRGTISASGDLSPLSYIAAAISGHPDSKVHXVHEGKEKILYAREAMALFNLEPVVLGPKEGLGLVNGTAVSASMATLALHDAHMLSLLSQSLTAMTVEAMVGHAGSFHPFLHDVTRPHPTQIEVAGNIRKLLEGSRFAVHHEEEVKVKDDEGILRQDRYPLRTSPQWLGPLVSDLIHAHAVLTIEAGQSTTDNPLIDVENKTSHHGGNFQAAAVANTMEKTRLGLAQIGKLNFTQLTEMLNAGMNRGLPSCLAAEDPSLSYHCKGLDIAAAAYTSELGHLANPVTTHVQPAEMANQAVNSLALISARRTTESNDVLSLLLATHLYCVLQAIDLRAXEFEFKKQFGPAIVSLIDQHFGSAMTGSNLRDELVEKVNKTLAKRLEQTNSYDLVPRWHDAFSFAAGTVVEVLSSTSLSLAAVNAWKVAAAESAISLTRQVRETFWSAASTSSPALSYLSPRTQILYAFVREELGVKARRGDVFLGKQEVTIGSNVSKIYEAIKSGRINNVLLKMLA

SEQ ID NO: 11 - protein, 716 aa, Rhodotorula glutinis mutant
  misc_feature (149)..(149): Xaa can be any naturally occurring amino acid
MAPSLDSISHSFANGVASAKQAVNGASTNLAVAGSHLPTTQVTQVDIVEKMLAAPTDSTLELDGYSLNLGDVVSAARKGRPVRVKDSDEIRSKIDKSVEFLRSQLSMSVYGVTTGFGGSADTRTEDAISLQKALLEHQLCGVLPSSFDXFRLGRGLENSLPLEVVRGAMTIRVNSLTRGHSAVRLVVLEALTNFLNHGITPXVPLRGTISASGDLSPLSYIAAAISGHPDSKVHVVHEGKEKILYAREAMALFNLEPVVLGPKEGLGLVNGTAVSASMATLALHDAHMLSLLSQSLTAMTVEAMVGHAGSFHPFLHDVTRPHPTQIEVAGNIRKLLEGSRFAVHHEEEVKVKDDEGILRQDRYPLRTSPQWLGPLVSDLIHAHAVLTIEAGQSTTDNPLIDVENKTSHHGGNFQAAAVANTMEKTRLGLAQIGKLNFTQLTEMLNAGMNRGLPSCLAAEDPSLSYHCKGLDIAAAAYTSELGHLANPVTTHVQPAEMANQAVNSLALISARRTTESNDVLSLLLATHLYCVLQAIDLRAXEFEFKKQFGPAIVSLIDQHFGSAMTGSNLRDELVEKVNKTLAKRLEQTNSYDLVPRWHDAFSFAAGTVVEVLSSTSLSLAAVNAWKVAAAESAISLTRQVRETFWSAASTSSPALSYLSPRTQILYAFVREELGVKARRGDVFLGKQEVTIGSNVSKIYEAIKSGRINNVLLKMLA

SEQ ID NO: 12 - protein, 716 aa, Rhodotorula glutinis mutant
  misc_feature (502)..(502): Xaa can be any naturally occurring amino acid
MAPSLDSISHSFANGVASAKQAVNGASTNLAVAGSHLPTTQVTQVDIVEKMLAAPTDSTLELDGYSLNLGDVVSAARKGRPVRVKDSDEIRSKIDKSVEFLRSQLSMSVYGVTTGFGGSADTRTEDAISLQKALLEHQLCGVLPSSFDSFRLGRGLENSLPLEVVRGAMTIRVNSLTRGHSAVRLVVLEALTNFLNHGITPIVPLRGTISASGDLSPLSYIAAAISGHPDSKVHVVHEGKEKILYAREAMALFNLEPVVLGPKEGLGLVNGTAVSASMATLALHDAHMLSLLSQSLTAMTVEAMVGHAGSFHPFLHDVTRPHPTQIEVAGNIRKLLEGSRFAVHHEEEVKVKDDEGILRQDRYPLRTSPQWLGPLVSDLIHAHAVLTIEAGQSTTDNPLIDVENKTSHHGGNFQAAAVANTMEKTRLGLAQIGKLNFTQLTEMLNAGMNRGLPSCLAAEDPSLSYHCKGLDIAAAAYTSELGHLANPVTTHVQPAEMANQAXNSLALISARRTTESNDVLSLLLATHLYCVLQAIDLRAXEFEFKKQFGPAIVSLIDQHFGSAMTGSNLRDELVEKVNKTLAKRLEQTNSYDLVPRWHDAFSFAAGTVVEVLSSTSLSLAAVNAWKVAAAESAISLTRQVRETFWSAASTSSPALSYLSPRTQILYAFVREELGVKARRGDVFLGKQEVTIGSNVSKIYEAIKSGRINNVLLKMLA

SEQ ID NO: 13 - DNA, 2148 bp, Rhodotorula glutinis
atggcaccctcgctcgactcgatctcgcactcgttcgcaaacggcgtcgcatccgcaaagcaggctgtcaatggcgcctcgaccaacctcgcagtcgcaggctcgcacctgcccacaacccaggtcacgcaggtcgacatcgtcgagaagatgctcgccgcgccgaccgactcgacgctcgaactcgacggctactcgctcaacctcggagacgtcgtctcggccgcgaggaagggcaggcctgtccgcgtcaaggacagcgacgagatccgctcaaagattgacaaatcggtcgagttcttgcgctcgcaactctccatgagcgtctacggcgtcacgactggatttggcggatccgcagacacccgcaccgaggacgccatctcgctccagaaggctctcctcgagcaccagctctgcggtgttctcccttcgtcgttcgactcgttccgcctcggccgcggtctcgagaactcgcttcccctcgaggttgttcgcggcgccatgacaatccgcgtcaacagcttgacccgcggccactcggctgtccgcctcgtcgtcctcgaggcgctcaccaacttcctcaaccacggcatcacccccatcgtccccctccgcggcaccatctctgcgtcgggcgacctctctcctctctcctacattgcagcggccatcagcggtcacccggacagcaaggtgcacgtcgtccacgagggcaaggagaagatcctgtacgcccgcgaggcgatggcgctcttcaacctcgagcccgtcgtcctcggcccgaaggaaggtctcggtctcgtcaacggcaccgccgtctcagcatcgatggccaccctcgctctgcacgacgcacacatgctctcgctcctctcgcagtcgctcacggccatgacggtcgaagcgatggtcggccacgccggctcgttccaccccttccttcacgacgtcacgcgccctcacccgacgcagatcgaagtcgcgggaaacatccgcaagctcctcgagggaagccgctttgctgtccaccatgaggaggaggtcaaggtcaaggacgacgagggcattctccgccaggaccgctaccccttgcgcacgtctcctcagtggctcggcccgctcgtcagcgacctcattcacgcccacgccgtcctcaccatcgaggccggccagtcgacgaccgacaaccctctcatcgacgtcgagaacaagacttcgcaccacggcggcaatttccaggctgccgctgtggccaacaccatggagaagactcgcctcgggctcgcccagatcggcaagctcaacttcacgcagctcaccgagatgctcaacgccggcatgaaccgcggcctcccctcctgcctcgcggccgaagacccctcgctctcctaccactgcaagggcctcgacatcgccgctgcggcgtacacctcggagttgggacacctcgccaaccctgtgacgacgcatgtccagccggctgagatggcgaaccaggcggtcaactcgcttgcgctcatctcggctcgtcgcacgaccgagtccaacgacgtcctttctctcctcctcgccacccacctctactgcgttctccaagccatcgacttgcgcgcgatcgagttcgagttcaagaagcagttcggcccagccatcgtctcgctcatcgaccagcactttggctccgccatgaccggctcgaacctgcgcgacgagctcgtcgagaaggtgaacaagacgctcgccaagcgcctcgagcagaccaactcgtacgacctcgtcccgcgctggcacgacgccttctccttcgccgccggcaccgtcgtcgaggtcctctcgtcgacgtcgctctcgctcgccgccgtcaacgcctggaaggtcgccgccgccgagtcggccatctcgctcacccgccaagtccgcgagaccttctggtccgccgcgtcgacctcgtcgcccgcgctctcgtacctctcgccgcgcactcagatcctctacgccttcgtccgcgaggagcttggcgtcaaggcccgccgcggagacgtcttcctcggcaagcaagaggtgacgatcggctcgaacgtctccaagatctacgaggccatcaagtcgggcaggatcaacaacgtcctcctcaagatgctcgct

SEQ ID NO: 14 - DNA, 2129 bp, Phanerochaete chrysosporium
atgccgccccttcaacagagcgcaagcccgaactcctggacaagttcgtcgaggcgtacgacgagctgcaatcctacagggaaggcaagccagtgatcgtcgacggacacaaccttagcatccccgctgtcgcagcgacggcgcgctttggcgccgcggtggccctcgacgagaaccccgagacccacgagcgcgtcctgcagagcagacgcgtgattgtcgacaaggtcagcactcagcgcagcgtatacggcgtcagcacgggcttcggcggatccggtacgttttgcgccgatacccgcacaagcgacccgctccagctcggccatgccctcctccagcaccagcacgtcggcgtgctgccgactcagaccgagtcgccgctcccagctctgcccctgggtgacccactcgcaacgacgagcatgccagaggcctgggttcgtggtgcaatccttatccgcatgaactcccttatccgggggcactctggtgtccgttgggagcttatcgagaagatgggcgagttgctgcgcgagaatatcaccccacttgttcccctgcgcggcagcatctctgcctcgggagatctctcgccgctctcatacattgccggaacgctcattggcagcccggctatccgtgtctttgatggccccgcctcctatggagcccgccgcatcctgccctcgaatatcgcgctcgccaaccacggtgtagccccgatcccgctctcttccaaggagcacttgggtattctcaacggcacagcattctccgcgtcggtcggcgcactggctctcaatgaggctgttcacctttctcttctcgcgcaggtctgcacggcaatgggcaccgaagcgatgattggtgcagttggctcgttcgacgcctttattcacgataccgctcgcccgcaccccggccaagtcgaggtcgctcgcaacgtccggactctcctcgaggactcgcagatggcggtcaaggctgaggacgaagttcacattgccgaggatgagggcgagctccgtcaggaccgctacccactccgcacggcggcacaattccttggcccccagattgaggacatcctgtctgcccacgagacggtcactcttgagtgcaactcgaccaccgataaccctcttattgatggcgagactggcaccgtgcaccatggtggtaacttccaggccatggctgtcacgaacgcgatggagaagacccgccttgccattcaccacattggcaagctgctctttgcccaggcaaccgagctcatcaaccccatgatgaaccgcggcctgccacccaacctcgcggcgactgacccgtcccacaactacttcgccaagggtatcgacattcacctcgctgcctacgtcggcgagctcggcttccttgccagcccggtctcttcgcacatccagtccgcggagatgcacaaccaggctgttaactcgctcgccctcgtgtctgctcgctacacgatcagcgcgctcgacgtcctctctctcctcacggcggcgtacctttacgtcctctgccaggcgctcgacctccgcgcgatgcacaacgacctccagtcgtcgctctcggcgatcgtccgcgagctgctgcccaagcatttcccgtctgcggcgaagcgcgcggacgctcttttgcccatcctcgagcgtaccatcttccgcgcactcaactcctcgagctcggcagactgcaaggcgcgcatggtcagcgtcgctgcctcgaccacgacgccgctcgtcgacttcctctctgcggacgctgcgctcgcatccgagctcgcgaacatcactgccttccgcacggacctcgcgacgcgcgctgccgatgcactcacgacgctgcgcacgcagtaccttgagggtgcccgtggcgcggcaccagcgagcaagtacctcggcaagacgcgccctgtgtacgagttcgtgcgtgtcaccctcaacgtgccgatgcacggccgcgagaacctgcacaacttcgagatgggcccgggtgtcgaggacggcatcatcggcaacaacatctcgacgatctacgaggccatccgcgacggcaagatgcagaacgtcgtcatgcagctcgtcaagtctatcaaggcgtag

SEQ ID NO: 15 - DNA, 2214 bp, Artificial Sequence
  Phanerochaete chrysosporium
atgccgtcccgtatcgattactatacttcttctggtaacggctacgcgcagagccgcaagtcctccgcgatctacccggctagcgcgtctaccggtcatgctgcaccgtccacggagcgcaagccggagctgctggacaagtttgttgaagcgtatgatgaactgcagtcttaccgtgaaggtaagccggttatcgttgacggccacaacctgtctatcccggcggtcgctgctactgcgcgttttggcgctgctgtggtactggacgaaaacccggaaactcacgaacgtgtcctgcagagccgccgtgtgattgtcgacaaagtttctacccagcgttctgtatacggcgttagcaccggcttcggcggttccgctgacactcgcacctctgacccgctgcaactgggccacgcgctgctgcagcaccaacacgtcggtgttctgccgactcagactgaaagcccgctgccagccctgccactgggtgacccgctggcgaccacctctatgccggaagcgtgggttcgcggtgctatcctgattcgtatgaactctctgatccgtggtcactctggcgttcgttgggaactgattgagaaaatgggtgagctgctgcgtgaaaacattaccccgctggtcccgctgcgtggtagcatctctgctagcggcgatctgtctccgctgtcctatatcgctggcaccctgattggttcccctgccattcgtgtgttcgacggtcctgcctcttacggtgcgcgtcgtatcctgccgtctaatattgcgctggccaaccacggtgtagcgccgatcccgctgtcttccaaagaacatctgggtattctgaatggtaccgctttttctgcctctgttggtgcactggctctgaacgaagctgttcacctgagcctgctggcgcaggtatgcaccgcaatgggtaccgaagcgatgatcggcgcagtgggcagctttgatgcgttcatccacgataccgctcgtccacacccgggccaggtagaggttgcgcgtaacgttcgtaccctgctggaagactctcagatggcagtcaaggcagaagatgaagtacatattgctgaagatgagggtgagctgcgccaagatcgttatccgctgcgtaccgctgcgcagttcctgggcccgcagatcgaagacattctgtctgcacacgagacggttaccctggaatgtaactccaccacggacaatccactgatcgacggcgaaactggcacggtacatcatggtggcaactttcaagctatggcggttaccaacgcaatggagaaaacccgtctggcgatccaccacatcggcaaactgctgttcgcgcaggccacggaactgatcaaccctatgatgaaccgcggcctgccgccgaacctggcagcaaccgaccctagccacaattacttcgcaaaaggtgttgatatccacctggcagcatacgttggcgagctgggtttcctggcgagcccagtttctagccacatccagtccgcagaaatgcataaccaggcagtgaactccctggctctggtttctgcgcgttacaccatttccgctctggatgtgctgtccctgctgaccgccgcatatctgtatgtcctgtgccaggccctggatctgcgtgcgatgcacaacgatctgcagtcctctctgtccgccatcgttcgtgaactgctgccgaagcattttccgtctgcggctaaacgcgcagatgcactgctgccgattctggaacgtaccatcttccgcgcactgaactcttcttcctccgcggactgcaaagcccgcatggtttccgtggcagcttctaccaccaccccgctggttgatttcctgagcgccgacgctgccctggcttccgaactggctaatattactgctttccgtaccgaactggcgacccgcgcagctgacgcactgaccaccctgcgcacccagtacctggaaggtgctcgcggtgccgcgccagcttctaaatatctgggcaaaactcgtccggtatacgaatttgtgcgtgtgactctgaacgtgccgatgcatggccgtgaaaatctgcacaacttcgaaatgggtccgggtgttgaggatggcatcatcggcaacaacatctccactatctacgaagccatccgtgatggtaaaatgcagaacgttgttatgcagctggttaagtctattaaagcctaa

SEQ ID NO: 16 - DNA, 32 bp, Artificial Sequence
  Primer for making thyA knockout F1 primer
cctagggtaccgctgctgctggaaggtgtggt

SEQ ID NO: 17 - DNA, 44 bp, Artificial Sequence
  Primer for making thyA knockout R2 primer
accgctaccttgaacgtgtcgcccatcttgcaggttaaaacgca

SEQ ID NO: 18 - DNA, 35 bp, Artificial Sequence
  Primer for making thyA knockout F2 primer
gaattctgactagtccctccctgataacgctgata

SEQ ID NO: 19 - DNA, 43 bp, Artificial Sequence
  Primer for making thyA knockout R1 primer
gcgacacgttcaaggtagcggtccgttaaattcttcgagacgc

SEQ ID NO: 20 - DNA, 42 bp, Artificial Sequence
  Primer for PCR amplification of the araC-araB region from E. coli strain FM5 (ATCC deposit no. 53911) genomic DNA
gccattcaggctgcgcattatgacaacttgacggctacatca

SEQ ID NO: 21 - DNA, 70 bp, Artificial Sequence
  Primer for PCR amplification of the araC-araB region from E. coli strain FM5 (ATCC deposit no. 53911) genomic DNA
agccaagcttggtacctcccggggagctccgaattcttcctcctgttagcccaaaaaacgggtatggaga

SEQ ID NO: 22 - DNA, 36 bp, Artificial Sequence
  Primer for PCR amplification of the transcription terminationsequences rrnBT1 and rrnBT2 from plasmid pTrc99A (Pharmacia Biotech, Amersham, GE Healthcare, Piscataway, NJ)
gcttgaaagcttggctgttttggcggatgagagaag

SEQ ID NO: 23 - DNA, 50 bp, Artificial Sequence
  Primer for PCR amplification of the transcription terminationsequences rrnBT1 and rrnBT2 from plasmid pTrc99A (Pharmacia Biotech, Amersham, GE Healthcare, Piscataway, NJ)
tcgggaattgaagcttaagagtttgtagaaacgcaaaaaggccatccgtc

SEQ ID NO: 24 - DNA, 46 bp, Artificial Sequence
  Oligonucleotides encoding for a linker sequence added to pLH312
ctacaaactcttggtaccccgtctagaactagtcaattcccgacag

SEQ ID NO: 25 - DNA, 46 bp, Artificial Sequence
  Oligonucleotides encoding for a linker sequence added to pLH312
ctgtcgggaattgactagttctagacggggtaccaagagtttgtag

SEQ ID NO: 26 - DNA, 53 bp, Artificial Sequence
  Primer for PCR amplification of the colE1 replication origin and rop (encodes a replication origin protein) gene locus of pBR322
ggacatcgcatgcgggttgccttactggttagcagaatgaatcaccgatacgc

SEQ ID NO: 27 - DNA, 53 bp, Artificial Sequence
  Primer for PCR amplification of the colE1 replication origin and rop (encodes a replication origin protein) gene locus of pBR322
ctagcggactagttctcatgaccaaaatcccttaacgtgagttttcgttccac

SEQ ID NO: 28 - DNA, 32 bp, Artificial Sequence
  Primer for PCR amplification the thyA locus of E. coli MG1655(ATCC # 47076), including the thyA coding region, the intrinsicthyA promoter located within the upstream gene umpA, and a transcriptional terminator in the downstream gene ppdA
cctagtctagagctgctgctggaaggtgtggt

SEQ ID NO: 29 - DNA, 35 bp, Artificial Sequence
  Primer for PCR amplification the thyA locus of E. coli MG1655(ATCC # 47076), including the thyA coding region, the intrinsicthyA promoter located within the upstream gene umpA, and a transcriptional terminator in the downstream gene ppdA
gaattctgactagtccctccctgataacgctgaga

(19) United States
(12) Patent Application Publication
(10) Pub. No.: US 2008/0014619 A1
(43) Pub. Date: Jan. 17, 2008

(54) Method of production of para-hydroxycinnamic acid

(76) Inventors: Lixuan Lisa Huang, Hockessin, DE (US); Tina K. Van Dyk, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/485,577

(22) Filed: Jul. 12, 2006

(51) Int. Cl. C12P 7/62; C12N 9/88; C12N 15/74; C12N 1/21

(52) U.S. Cl. 435/135; 435/232; 435/252.33; 435/488

(57) ABSTRACT

Methods for the production of PHCA are disclosed. The methods rely on the tight gene expression of genetic constructs encoding polypeptides having tyrosine ammonia lyase activity. Promoters of choice are arabinose inducible.

### FIELD OF INVENTION

The invention relates to the field of microbiology and the production of p-hydroxycinnamic acid (pHCA). More specifically, tight regulation of the expression of a gene encoding tyrosine ammonia lyase results in increased levels of tyrosine ammonia lyase production and enhanced p-hydroxycinnamic acid production.

### BACKGROUND OF INVENTION

Production of chemicals from microorganisms has been an important application of biotechnology. Typically, the step in developing such a bio-production method may include: 1) selection of a proper microorganism host, 2) elimination of metabolic pathways leading to by-products, 3) deregulation of such pathways at both enzyme activity level and the transcriptional level, and 4) over-expression of appropriate enzymes in the desired pathways. In addition, some manipulations at the molecular level are required to control expression of particular enzyme(s) in order to prevent accumulation of toxic end-products. The present invention uses tightly regulated promoters for over-expression of an enzyme to control production of the toxic desired product pHCA.

pHCA is a useful monomer for the production of Liquid Crystal Polymers (LCP). LCPs are used in liquid crystal displays, and in high speed connectors and flexible circuits for electronic, telecommunication, and aerospace applications. Because of their resistance to sterilizing radiation and their high oxygen and water vapor barrier properties, LCPs are used in medical devices, and in chemical and food packaging.

Chemical synthesis of pHCA is known (see for example JP 2004231541 and JP 200414943). Additionally pHCA, an intermediate in the lignin biosynthetic pathway is commonly extracted from plant tissue [Plant Biochemistry, Ed. P. M. Dey, Academic Press, (1997)] and methods of its isolation and purification of are known [R. Benrief, et al., Phytochemistry, 47, 825-832; WO 972134; Bartolome et al., Journal of the Science of Food and Agriculture (1999), 79(3), 435-439. These methods are time consuming, expensive and cumbersome. A more facile method of production is therefore needed for the inexpensive and large scale synthesis of this monomer. A fermentation route offers one possible solution.

Biological production of pHCA is known (see for example commonly owned U.S. Pat. No. 6,368,837; U.S. Ser. No. 10/138970 and U.S. Ser. No. 11/105259). In these disclosures pHCA is produced in recombinant microbes via the conversion of phenylalanine to cinnamic acid (CA) by an enzyme known as phenylalanine ammonia lyase (PAL), which is in turn converted to pHCA via an- enzyme system comprising cinnamate-4-hydroxylase (C4H) and a cytochrome P450-dependent monooxygenase (P450). Alternatively pHCA may be produced directly from tyrosine via enzymatic catalysis by tyrosine ammonia lyase (TAL), an enzyme activity related functionally and structurally to PAL.

Although pHCA is naturally produced in plants it is not native to most microbes. Consequently, one of the difficulties in the microbial production of pHCA is toxicity of pHCA to the microbial host. Regulating the production of pHCA in a fermentation is key to maximizing production. One solution to this difficulty would be to regulate the expression of the genes encoding the relevant enzymes (i.e. PAL or TAL) for pHCA production in a manner so as to reduce the effect of toxicity. Typically this type of genetic control is managed through the use of regulated or includible promoters which may be used to differentially drive gene expression.

Examples of such regulated or inducible promoters include: the P_{L} promoter of the lambda phage of E. coli, the promoter of the tryptophan operon of E. coli, the lactose operon of E. coli. Also, fusion promoters composed of the trp promoter and the lac promoter are being widely used for such purposes. These are all “defined” as tightly regulated promoters in the literature, however their expression may be leaky in the un-induced state. While some of these promoters can be induced by the commonly used inducer, isopropylthio-β-D-galactoside (IPTG), others can be engineered to accept IPTG as the inducer. However, use of IPTG is not practical for large-scale bioprocesses due to its high cost. In addition, higher concentrations of IPTG increase the cells' metabolic burden resulting in reduction of maximal expression of the target gene [Donovan et al., J. Ind. Microbiol. 16:145-154 (1996)].

The arabinose inducible promoter (araB), which is regulated by catabolite repression, is both positively and negatively regulated by the transcriptional regulator AraC and is therefore tightly regulated. Replacing glucose with arabinose in cultures can therefore lead to a highly increased level of transcription of the target gene(s). A variety of expression vectors have been developed based on the Salmonella typhimurium araB promoter [Cagnon et al, Protein Engineering, vol 4(7), pp 843-847, (1991)] and [Guzman et al J. Bacteriol (1995), vol 177(14), pp 4141-30, (1995)]. The use of araB promoter has also been used to effect the tight regulation of gene expression (Wild et al., Methods in Molecular Biology (Totowa, N.J., United States) (2004), 267(Recombinant Gene Expression (2nd Edition)), 155-167; Boomershine et al in Protein Expression and Purification, vol 28(2), pp 246-251, (2003)) and its use to mitigate toxicity of hydroxycinnamates has been appreciated (Parke, et al., Applied and Environmental Microbiology (2004), 70(5), 2974-2983). Similar to the araB promoter, the rhamnose-inducible promoter (rhaB) provides tightly regulated gene expression that is inducible by addition of rhamnose [Moralejo et al, J. Bacteriol. vol 175(17), pp 5584-94, (1993]) and [Haldimann et al, J. Bacteriol. vol 180(5), pp 1277-86 (1998.); Cardona et al., Plasmid (2005), 54(3), 219-228].

Although tight gene regulation is described, it has not yet been effectively applied to solve the problem of pHCA toxicity in the fermentive production of pHCA. There remains a need therefore for a microbial system, useful for the production of pHCA, where the relevant genes controlling expression of enzymes for pHCA synthesis are sufficiently regulated to control product toxicity.

Therefore there is a need for an expression system with a tightly regulated promoter that allows for efficient gene expression control using a readily available and inexpensive inducer.

Applicants have solved the stated problem by constructing an expression system for TAL production, which is regulated tightly by the araB promoter. This system provides minimal expression when not induced and strong specific expression when induced. This makes the araB system particularly well suited for producing TALactivity in E. coli.

### SUMMARY OF INVENTION

The present invention relates to the production of pHCA using bacterial hosts having a genetic construct encoding a polypeptide having tyrosine ammonia lyase (TAL) activity whose expression is tightly regulated to control production of toxic pHCA. The bacterial host may contain either an endogenous source of tyrosine, thus independently making pHCA, or may utilize tyrosine supplied exogenously. In either case tight regulation of the TAL expression is necessary to prevent the premature formation of pHCA which is toxic to the host organism. The present method is effective in part because the inducible promoter remains “switched-off” during cell propagation, when gene expression is unnecessary and undesirable, and is only “switched on” in the presence of a specific inducer, to provide high levels of gene expression during production.

Accordingly the invention provides an E. coli K12 bacterial production host comprising at least one genetic construct encoding a polypeptide having tyrosine ammonia lyase activity, operably linked to a tightly regulated inducible promoter. Preferred inducible promoters are araB, and rhaB.

In another embodiment the invention provides a method for the production of tyrosine ammonia lyase comprising:
a) providing an E. coli K12 bacterial production host comprising at least one genetic construct encoding a polypeptide having tyrosine ammonia lyase activity operably linked to a tightly regulated inducible promoter selected from the group consisting of araB and rhaB;
b) growing the E. coli K12 bacterial production host of (a) in a growth medium; and
c) inducing the inducible promoter of (a) whereby tyrosine ammonia lyase is produced.

In another embodiment the invention provides a method for the production of p-hydroxycinnamic acid comprising:

a) providing a source of tyrosine;

b) growing an E. coli K12 bacterial host comprising at least one genetic construct encoding a polypeptide having tyrosine ammonia lyase activity operably linked to a tightly regulated inducible promoter;

c) inducing the inducible promoter of b) whereby tyrosine ammonia lyase is produced in the E. coli K12 bacterial host;

d) combining the tyrosine of step (a) with the E. coli K12 bacterial host of step c) whereby p-hydroxycinnamic acid is produced; and

e) optionally recovering the p-hydroxycinnamic acid.

In an alternate embodiment the invention provides a method for the production of p-hydroxycinnamic acid comprising:

a) providing an E. coli K12 bacterial host comprising:
i) at least one genetic construct encoding a polypeptide having tyrosine ammonia lyase activity operably linked to a tightly regulated inducible promoter; and
ii) an endogenous source of tyrosine;

b) growing the E. coli K12 bacterial host of (a) under conditions whereby tyrosine is produced in the absence of tyrosine ammonia lyase activity;

c) inducing the inducible promoter of step (a)(i) where in the at least one genetic construct expresses the polypeptide having tyrosine ammonia lyase activity whereby p-hydroxycinnamic acid is produced; and

d) optionally recovering the p-hydroxycinnamic acid of step (c).

In another variation the invention provides A method for the production of p-hydroxycinnamic acid comprising:

a) providing an E. coli K12 bacterial host comprising at least one genetic construct encoding a polypeptide having tyrosine ammonia lyase activity operably linked to a tightly regulated inducible promoter;

b) providing a bacterial production host that is overproducing for tyrosine;

c) cofermenting the E. coli K12 bacterial host of step (a) and the bacterial tyrosine overproducing host of step (b) in a single fermentation phase;

d) Inducing the cofermented E. coli K12 bacterial host of step (c) whereby tyrosine ammonia lyase is expressed and p-hydroxycinnamic acid is produced; and

d) optionally recovering the p-hydroxycinnamic acid of step (d).

In another embodiment the invention provides A method for the production of p-hydroxycinnamic acid comprising:

a) growing an E. coli K12 bacterial host in a growth medium comprising at least one genetic construct encoding a polypeptide having tyrosine ammonia lyase activity operably linked to a tightly regulated inducible promoter;

b) providing tyrosine in the growth medium of (a);

c) inducing the inducible promoter whereby tyrosine ammonia lyase is expressed and p-hydroxycinnamic acid is produced; and

d) optionally recovering the p-hydroxycinnamic acid of step (c).

In another embodiment the invention provides an E. coli K12 bacterial production host comprising;
a) at least one genetic construct encoding a polypeptide having tyrosine ammonia lyase activity operably linked to a tightly regulated inducible promoter; and
b) an endogenous source of tyrosine.

### BRIEF DESCRIPTION OF THE FIGURES, BIOLOGICAL DEPOSITS AND SEQUENCE DESCRIPTIONS

FIG. 1 is an illustration of the aromatic amino acid biosynthetic pathway.

FIG. 2 is plasmid map of pLH312.

FIG. 3 is plasmid map of pLH320.

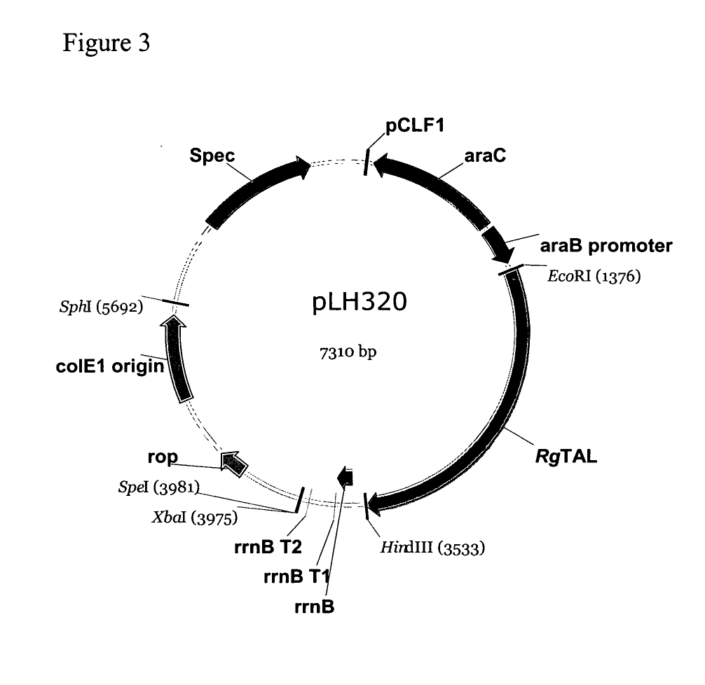

FIG. 4 shows a polyacrylamide gel separation of insoluble (I) and soluble (S) protein extracts from DPD5142 cells induced with different amounts of arabinose.

FIG. 5 is a plasmid map of pLH330.

FIG. 6 shows a graph of TAL activity in strain DPD5124 at different times after arabinose induction.

FIG. 7 shows a polyacrylamide gel separation of insoluble (I) and soluble (S) protein extracts from DPD5142 cells induced at different times after arabinose induction.

The invention can be more fully understood from the following detailed description and the accompanying sequence descriptions which form a part of this application.

The following sequences conform with 37 C.F.R. 1.821-1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

A Sequence Listing is provided herewith on Compact Disk. The contents of the Compact Disk containing the Sequence Listing are hereby incorporated by reference in compliance with 37 CFR 1.52(e). The Compact Disks are submitted in triplicate and are identical to one another. The disks are labeled “Copy 1—Sequence Listing”, “Copy 2—Sequence listing”, and CRF—Sequence Listing. The disks contain the following file: CL3282.ST25 having the following size: 85,000 bytes and which were created Jul. 10, 2006.

SEQ ID NO:1 is the amino acid sequence of the Rhodotorula glutinis TAL protein.

SEQ ID NO:2 is the amino acid sequence of the Phanerochaete chrysosporium TAL protein.

SEQ ID NO:3 is the amino acid sequence of the Trichosporon cutaneum PAL/TAL protein.

SEQ ID NO:4 is the amino acid sequence of the Rhodobacter sphaeroides PAL/TAL protein.

SEQ ID NO:5 is the amino acid sequence of the Ustilago maydis PAL/TAL protein.

SEQ ID NO:6 is the amino acid sequence of the Petroselinum crispum PAL/TAL protein.

SEQ ID NO:7 is the amino acid of the mutant R. glutinis PAL enzyme having enhanced TAL activity.

SEQ ID NO:8 is the amino acid sequence encoded of the mutant TAL enzyme identified as RM 120-1.

SEQ ID NO:9 is the amino acid sequence encoded of the mutant TAL enzyme identified as RM120-2.

SEQ ID NO:10 is the amino acid sequence encoded of the mutant TAL enzyme identified as RM120-4.

SEQ ID NO:11 is the amino acid sequence encoded of the mutant TAL enzyme identified as RM120-7.

SEQ ID NO:12 is the amino acid sequence encoded of the mutant TAL enzyme identified as RM492-1.

SEQ ID NO:13 is the DNA sequence of the coding region for Rhodotorula glutinis TAL.

SEQ ID NO:14 is the DNA sequence of the coding region for Phanerochaete chrysosporium TAL.

SEQ ID NO:15 is the E. coli codon optiomized DNA sequence encoding Phanerochaete chrysosporium TAL.

SEQ ID NOs:16-19 are primers for making a thyS knockout strain.

SEQ ID NOs:20 and 21 are primers for PCR amplification of the araC-araB region from E. coli strain FM5 (ATCC deposit no. 53911) genomic DNA.

SEQ ID NOs:22 and 23 are primers for PCR amplification of the transcription termination sequences rrnBT1 and rrnBT2 from plasmid pTrc99A (Pharmacia Biotech, Amersham, GE Healthcare, Piscataway, N.J.).

SEQ ID NOs:24 and 25 are oligonucleotides of a linker sequence added to pLH312.

SEQ ID NOs:26 and 27 are primers for PCR amplification of the colE1 replication origin and rop (encodes a replication origin protein) gene locus of pBR322.

SEQ ID NOs:28 and 29 are primers for PCR amplification the thyA locus of E. coli MG1655 (ATCC #47076), including the thyA coding region, the intrinsic thyA promoter located within the upstream gene umpA, and a transcriptional terminator in the downstream gene ppdA.

### DETAILED DESCRIPTION OF INVENTION

The present invention describes biological methods for production of p-hydroxycinnamic acid (pHCA) through the use of E. coli whole cell biocatalysts demonstrating tightly regulated expression of high levels of tyrosine ammonia lyase (TAL). E. coli cells are transformed with a plasmid containing a TAL gene. These biocatalysts can be used for the conversion of tyrosine to p-hydroxycinnamic acid (pHCA).

pHCA is a useful monomer for production of Liquid Crystal Polymers (LCP). LCPs are polymers that exhibit an intermediate or mesophase between the glass-transition temperature and the transition temperature to the isotropic liquid or have at least one mesophase for certain ranges of concentration and temperature. The molecules in these mesophases behave like liquids and flow, but also exhibit the anisotropic properties of crystals. LCPs are used in liquid crystal displays, and in high speed connectors and flexible circuits for electronic, telecommunication, and aerospace applications. Because of their resistance to sterilizing radiation and their high oxygen and water vapor barrier properties, LCPs are used in medical devices, and in chemical and food packaging.

The following abbreviations and definitions will be used for the interpretation of the specification and the claims.

“Phenyl ammonia-lyase” is abbreviated PAL.

“Tyrosine ammonia-lyase” is abbreviated TAL.

“para-hydroxycinnamic acid” or “p-hydroxycinnamic” is abbreviated pHCA. “Cinnamate 4-hydroxylase” is abbreviated C4H.

As used herein the terms “cinnamic acid” and “cinnamate” are used interchangeably.

The term “TAL activity” refers to the ability of a protein to catalyze the direct conversion of tyrosine to pHCA.

The term “PAL activity” refers to the ability of a protein to catalyze the conversion of phenylalanine to cinnamic acid.

The term “PAL/TAL activity” or “PAL/TAL enzyme” refers to a protein which contains both PAL and TAL activity. Such a protein has at least some specificity for both tyrosine and phenylalanine as an enzymatic substrate.

The term “invention” or “present invention” as used herein shall not be limited to any particular embodiment of the invention but shall refer to all the varied embodiments described by the specification ad the claims.

“Gene” refers to a nucleic acid fragment that expresses a specific protein, including regulatory sequences preceding (5′ non-coding sequences) and following (3′ non-coding sequences) the coding sequence. “Native gene” or “wild type gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes.

The term “genetic construct” refers to a nucleic acid fragment that encodes for expression of one or more specific proteins. In the gene construct the gene may be native, chimeric, or foreign in nature. Typically a genetic construct will comprise a “coding sequence”. A “coding sequence” refers to a DNA sequence that codes for a specific amino acid sequence. “Promoter” or “Initiation control regions” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”. “Inducible promoters” on the other hand are not always active the way constitutive promoters are. They can be induced by particular environmental conditions. It is further recognized that since in most cases the exact boundaries of regulatory sequences have not been completely defined, DNA fragments of different lengths may have identical promoter activity.

The term “expression”, as used herein, refers to the transcription and stable accumulation of sense (mRNA) or antisense RNA derived from the nucleic acid fragment of the invention. Expression may also refer to translation of mRNA into a polypeptide.

The term “over-expression” as used herein, refers to the production of a gene product in transgenic organisms that exceeds levels of production in normal or non-transformed organisms.

The term “Messenger RNA (mRNA)” as used herein, refers to the RNA that is without introns and that can be translated into protein by the cell.

The term “transformation” as used herein, refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The terms “plasmid” and “vector” as used herein, refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The term “host cell” is meant that a cell that contains a plasmid or a vector and supports the replication or expression of the plasmid or the vector.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “selectable marker” means an identifying factor, usually an antibiotic or chemical resistance gene, that is able to be selected for based upon the marker gene's effect, i.e., resistance to an antibiotic, wherein the effect is used to track the inheritance of a nucleic acid of interest and/or to identify a cell or organism that has inherited the nucleic acid of interest.

The term “pHCA production strain” will refer to a microbial strain, typically an enteric bacteria,that is engineered to contain a genetic construct encoding a polypeptide having tyrosine ammonia lyase activity, with the coding region under the control of a tightly regulated promoter, typically, araB or rhaB.

The term “tyrosine over-producing strain” refers to a microbial strain that produces endogenous levels of tyrosine that are significantly higher than those seen in the wildtype of that strain. When produced at high levels, tyrosine is typically excreted into the medium, and thus a tyrosine over-producing strain is generally also a “tyrosine excreting strain”.

The term “amino acid” will refer to the basic chemical structural unit of a protein or polypeptide. The following abbreviations will be used herein to identify specific amino acids:

| | Three- | One- |
|---|---|---|
| | Letter | Letter |
| Amino Acid | Abbreviation | Abbreviation |
| Alanine | Ala | A |
| Arginine | Arg | R |
| Asparagine | Asn | N |
| Aspartic acid | Asp | D |
| Asparagine or aspartic acid | Asx | B |
| Cysteine | Cys | C |
| Glutamine | Gln | Q |
| Glutamine acid | Glu | E |
| Glutamine or glutamic acid | Glx | Z |
| Glycine | Gly | G |
| Histidine | His | H |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Standard recombinant DNA and molecular cloning techniques used here are well known in the art and are described by Sambrook, J., Fritsch, E. F. and Maniatis, T. Molecular Cloning: A Laboratory Manual, 2^{nd} ed.; Cold Spring Harbor Laboratory: Cold Spring Harbor, N.Y., 1989 (hereinafter “Maniatis”); and by Silhavy, T. J., Bennan, M. L. and Enquist, L. W. Experiments with Gene Fusions; Cold Spring Harbor Laboratory: Cold Spring Harbor, N.Y., 1984; and by Ausubel, F. M. et al., In Current Protocols in Molecular Biology, published by Greene Publishing and Wiley-lnterscience, 1987.

The present invention relates to methods for production of pHCA using a system having tight regulation of genetic constructs that encode polypeptides having TAL activity. pHCA will be formed in the presence of the TAL enzyme where sufficient tyrosine is present. Tyrosine may be produced endogenously by a cell having the TAL encoding constructs or supplied exogenously either in purified or partially purified form or by another bacterial cell producing tyrosine. The challenge presented by the production of pHCA using the methods described herein is the toxicity of pHCA to the producing host. Where tyrosine and active TAL are present, pHCA will be formed. If pHCA is produced early in the growth phase of the cultures it will inhibit the host cell and damage product yields.

There are several possible methods for pHCA production contemplated herein. In one embodiment the host cell producing TAL may also comprise a biosynthetic pathway for high levels of tyrosine production. In this situation if TAL expression is not regulated, pHCA would immediately accumulate damaging the host cell. Placing the TAL gene under the control of a regulated, inducible promoter will facilitate induction of the gene only after the culture has attained adequate growth and tyrosine has accumulated. In another embodiment the host cell producing TAL may receive tyrosine exogenously via addition to the TAL cells. Again, regulation of TAL expression is necessary to prohibit pHCA formation until the culture has attained adequate growth. Finally, the host cell producing TAL may be co-fermented with another cell having a biosynthetic pathway for tyrosine production. Again, having the ability to induce expression of TAL at the appropriate time will avoid pHCA toxicity during the culture growth phase.

Applicants have discovered that Escherichia host cell toxicity to pHCA can be very pronounced and even tight regulation of TAL expression by some promoters will still allow for enough pHCA to be formed to negatively affect the growth the cultures. Surprisingly, applicants have discovered that the araB promoter was very effective at controlling leaky expression of this gene and preventing pHCA production early in the culture growth cycle.

### Genes Encoding Tyrosine Ammonia Lyase (TAL) Activity and Phenylalanine Ammonia Lyase (PAL) Activity.

The present invention makes use of the enzymes having tyrosine ammonia lyase (TAL) activity. Enzymes having this activity generally also have phenylalanine ammonia lyase (PAL) activity. The PAL enzyme acts on phenylalanine for the production of cinnamic acid. Cinnamic acid may be converted to pHCA via cytochrome p-450/reductase enzymes system. The TAL enzyme activity directly converts tyrosine to pHCA. This reaction to pHCA is more energy efficient and requires less genetic engineering than the route from phenylalanine, and is therefore preferred.

Most PAL enzymes have a substrate specificity for phenylalanine, but will also use tyrosine for the direct conversion to pHCA. There are no known genes which encode an enzyme having exclusively TAL activity, i.e., which will use only tyrosine as the substrate for production of pHCA. Genes encoding PAL are known in the art and several have been sequenced from both plant and microbial sources (see for example EP 321488 [R. toruloides]; WO 9811205 [Eucalyptus grandis and Pinus radiata]; WO 9732023 [Petunia]; JP 05153978 [Pisum sativum]; WO 9307279 [potato, rice]). The sequence of PAL genes is available (see for example GenBank AJ010143 and X75967).

Where expression of a wild type PAL gene in a recombinant host is desired, the wild type gene may be obtained from any source including but not limited to, yeasts such as Rhodotorula sp., Rhodosporidium sp. and Sporobolomyces sp.; bacterial organisms such as Streptomyces; and plants such as pea, potato, rice, eucalyptus, pine, corn, petunia, arabidopsis, tobacco, and parsley. Within the context of the present invention genes isolated from the organisms Rhodotorula sp., Rhodospoddium sp., Rhodotorula glutinis, and Sporobolomyces sp. are preferred.

Several of the PAL enzymes mentioned above can use tyrosine as well as phenylalanine as substrate. Such enzymes will be referred to herein as PAL/TAL enzymes or activities and therefore their genes are referred to as pal/tal or tal genes. For example, the PAL enzyme isolated from parsley [Appert et al., Eur. J. Biochem. 225:491 (1994)] and corn [Havir et al., Plant Physiol. 48:130 (1971)] and the PAL enzyme isolated from the yeast Rhodosporidium [also called Rhodotorula; Hodgins D. S., J. Biol. Chem. 246:2977 (1971)] demonstrate TAL activity. Where it is desired to create a recombinant organism expressing a wild type gene encoding PAL/TAL activity, genes isolated from, for example, maize, wheat, parsley, Rhizoctonia solani, Rhodosporidium, Sporobolomyces pararoseus and Rhodosporidium may be used as discussed in Hanson and Havir, [The Biochemistry of Plants; Academic: N.Y., 1981; Vol. 7, pp 577-625]. Mutant enzymes having enhanced TAL activity have also been reported (see for example commonly owned U.S. Pat No. 6,368,837 and U.S. Pat. No. 6,521,748, herein incorporated by reference) and are particularly useful sources of TAL enzyme for use in the present invention.

Methods of obtaining these or homologous wild type genes using sequence-dependent protocols are well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction (PCR), ligase chain reaction (LCR)).

For example, genes encoding homologs for any one of the mentioned activities (PAL/TAL or TAL ) could be isolated directly by using all or a portion of the known sequences as DNA hybridization probes to screen libraries from any desired plant, fungi, yeast, or bacteria using methodology well known to those skilled in the art. Specific oligonucleotide probes based upon the literature nucleic acid sequences can be designed and synthesized by methods known in the art (Maniatis, supra). Moreover, the entire sequences can be used directly to synthesize DNA probes by methods known to the skilled artisan such as random primers DNA labeling, nick translation, or end-labeling techniques, or RNA probes using available in vitro transcription systems. In addition, specific primers can be designed and used to amplify a part of or full-length of the instant sequences. The resulting amplification products can be labeled directly during amplification reactions or labeled after amplification reactions, and used as probes to isolate full length cDNA or genomic fragments under conditions of appropriate stringency.

In addition, two short segments of the literature sequences may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the literature sequences, and the sequence of the other primer takes advantage of the presence of the polyadenylic acid tracts to the 3′ end of the mRNA precursor encoding bacterial genes. Alternatively, the second primer sequence may be based upon sequences derived from the cloning vector. For example, the skilled artisan can follow the RACE protocol (Frohman et al., PNAS USA 85:8998 (1988)) to generate cDNAs by using PCR to amplify copies of the region between a single point in the transcript and the 3′ or 5′ end. Primers oriented in the 3′ and 5′ directions can be designed from the literature sequences. Using commercially available 3′ RACE or 5′ RACE systems (Invitrogen, Carlsbad, Calif.), specific 3′ or 5′ cDNA fragments can be isolated (Ohara et al., PNAS USA 86:5673 (1989); Loh et al., Science 243:217 (1989)).

The skilled person will recognize that, for the purposes of the present invention, enzymes with TAL activity from any source will be suitable. TAL enzymes that may be used include, but are not limited to, those from Rhodotorula glutinis (SEQ ID NO;1; U.S. Pat. No. 6,521,748), Phanerochaete chrysosporium (SEQ ID NO:2; disclosed in co-owned and co-pending U.S. application CL3434), Trichosporon cutaneum (SEQ ID NO:3; U.S. Pat. No. 6,951,751), Rhodobacter sphaeroides (SEQ ID NO:4; US20040059103), Ustilago maydis (SEQ ID NO:5; Kim et al. (2001) Curr. Genet. 40:40-48), and parsley (SEQ ID NO:6; (Lois et al. (1989) JOURNAL EMBO J. 8:1641-1648). In addition to natural tyrosine/phenylalanine ammonia lyases, altered enzymes may be used such as a mutagenized Rhodosporidium toruloides (R. glutinis) enzyme with an increased TAL/PAL activity ratio over that of the wild type enzyme (SEQ ID NO:7; U.S. Pat. No. 6,368,837), and several other mutant enzymes with enhanced TAL activities (SEQ ID NOs:8, 9, 10, 11, 12; U.S. Pat. No. 6,521,748) Several of these enzymes with high TAL activity have been introduced into microorganisms for production of pHCA (U.S. Pat. No. 6,368,837, US20040059103 A1). Particularly suitable are the TAL proteins of Rhodotorula glutinis and Phanerochaete chrysosporium. Preferred in this invention is the TAL protein from Rhodotorula glutinis as set forth in SEQ ID NO:1. DNA sequences encoding the desired TAL protein may be the natural coding sequence, or a synthetic, optionally codon optimized, coding sequence that translates to the amino acids of the selected protein. Particularly suitable are the natural sequences encoding RgTAL (SEQ ID NO:13) and PcTAL (SEQ ID NO:14), as well as a codon-optimized sequence encoding PcTAL (SEQ ID NO:15; U.S. application CL3434).

### Promoters

An important aspect of the invention is the selection of a promoter that will regulate the expression of the genetic construct expressing the TAL enzyme sufficiently well so as to prevent the premature production of pHCA.

Virtually any promoter that tightly regulates the expression of these genes in an E. coli host is suitable for the present invention. However, for the purpose of this invention inducible promoters are preferred. Examples of inducible promoters, which may be selected, include: araB, rhaB, and other very tightly regulated promoters such as a tac promoter with two lacl operator sequences after the promoter. Arabinose inducible promoters are particularly useful and a wide variety are known in the art. It is contemplated that any will be useful in the present invention. The following is a non-limiting list of genomes containing arabinose inducible promoters useful herein:

Escherichia coli K12, complete genome gi|49175990|ref|NC_{—}000913.21|[49175990]

Yersinia pestis KIM, complete genome gi|22002119|gb|AE009952.1|[22002119]

Lactobacillus sakei strain 23K complete genome gi|78609255|emb|CR936503.1|[78609255]

Salmonella typhimurium LT2, complete genome gi|16763390|ref|NC_{—}003197.1|[16763390]

Shigella flexneri 2a str. 301, complete genome gi|24111450|ref|NC_{—}004337.1|[24111450]

For the purpose of this invention, the araB promoter is the most preferred promoter. The araB promoter remains “switched-off” during cell propagation, when gene expression is unnecessary and undesirable. However, during production when a high level of gene expression is required, the promoter is “switched on” by the inducer arabinose. Therefore expression of TAL is regulated tightly by the araB promoter since it provides minimal expression when uninduced and strong specific expression when induced. The araB promoter is operably linked to the TAL coding region from R. glutinis and is cloned into a plasmid that replicates in E. coli.

Methods of obtaining these promoters using sequence-dependent protocols are well known in the art. Examples of sequence-dependent protocols include, but are not limited to, methods of nucleic acid hybridization, and methods of DNA and RNA amplification as exemplified by various uses of nucleic acid amplification technologies, e.g., PCR and LCR.

The use of the above tightly regulated promoters under inducing conditions is particularly effective for the expression and production of TAL, resulting in host cells producing TAL at concentrations of about 10% to about 20% total soluble cellular protein, where about 20% to about 50% total soluble cellular protein is anticipated and about 50% to about 70% total soluble cellular protein is expected.

Construction of The Plasmid Vector

Expression of genetic constructs encoding polypeptides having TAL activity will generally require the construction of an expression vector carrying the genetic construct. Background vectors useful for the transformation of suitable microbial host cells are well known in the art and many are commercially available. Typically the vector contains sequences directing transcription and translation of the relevant gene, a selectable marker, and sequences allowing autonomous replication or chromosomal integration. Suitable vectors comprise a region 5′ of the coding region which harbors transcriptional initiation controls and a region 3′ of the coding region which controls transcriptional termination. It is most preferred when both control regions are derived from genes homologous to the transformed host cell, although it is to be understood that such control regions need not be derived from the genes native to the specific species chosen as a production host.In addition a translation leader sequence may be included for TAL expression control. The skilled person will be able to pair promoters and terminators as appropriate.

Markers will be useful in the vector for screening purposes. A selection marker may be used to identify only those cells that receive the plasmid vector. Types of usable markers include nutritional, screenable, and selection markers. Many different selection markers available for bacterial cell selection may be used, including nutritional markers, antibiotic resistance markers, and metabolic markers. Some specific examples include, but are not limited to, thyA, serA, ampicillin resistance, kanamycin resistance, carbenicillin resistance, spectinomycin resistance, and the alanine racemase gene.

### PHCA Production Hosts

The production host of the present invention will include any organism in which the tightly regulated promoter selected for TAL expression is properly functional such that induced TAL expression leads to high levels of TAL activity in the host cells.

Particularly suitable in the present invention are members of the enteric class of bacteria. Enteric bacteria are members of the family Enterobacteriaceae and include such members as Escheichia, Salmonella, and Shigella. They are gram-negative straight rods, 0.3-1.0×1.0-6.0 mm, motile by peritrichous flagella (except for Tatumella) or nonmotile. They grow in the presence and absence of oxygen and grow well on peptone, meat extract, and (usually) MacConkey's media. Some grow on D-glucose as the sole source of carbon, whereas others require vitamins and/or mineral(s). They are chemoorganotrophic with respiratory and fermentative metabolism but are not halophilic. Acid and often visible gas is produced during fermentation of D-glucose, other carbohydrates, and polyhydroxyl alcohols. They are oxidase negative and, with the exception of Shigella dysenteriae 0 group 1 and Xenorhabdus nematophilus, catalase positive. Nitrate is reduced to nitrite (except by some strains of Erwinia and Yersina). The G+C content of DNA is 38-60 mol % (Tₘ, Bd). DNAs from species within most genera are at least 20% related to one another and to Eschedichia coli, the type species of the family. Notable exceptions are species of Yersina, Proteus, Providenica, Hafnia and Edwardsiella, whose DNAs are 10-20% related to those of species from other genera. Except for Erwinia chrysanthemi, all species tested contain the enterobacterial common antigen (Bergy's Manual of Systematic Bacteriology, D. H. Bergy et al., Baltimore: Williams and Wilkins, 1984). Preferred enteric bacteria for use in the present invention will be E. coli and particularly, E. coli k12 or E. coli B.

Within the context of the present invention the enteric host will need to meet several requirements. Because of the preferred use of arabinose inducible promoters, typically the host will be any E. coli strain that cannot metabolize arabinose. Where the host cell is not naturally unable to metabolize arabinose, it may be easily modified. Typically this can be accomplished by deleting the araBAD operon in the chromosome of the E. coli host through various approaches of gene knockout or disruption. Where sequence of the gene to be disrupted is known, one of the most effective methods of gene disruption is where foreign DNA is inserted into a structural gene so as to disrupt transcription. This can be effected by the creation of genetic cassettes comprising the DNA to be inserted (often a genetic marker) flanked by sequence having a high degree of homology to a portion of the gene to be disrupted. Introduction of the cassette into the host cell results in insertion of the foreign DNA into the structural gene via the native DNA replication mechanisms of the cell. (See for example [Datsenko K A et al., Proc. Natl. Acad. Sci. USA 97: 6640-6645 (2000); Hamilton et al. (1989) J. Bacteriol. 171:4617-4622, Balbas et al. (1993) Gene 136:211-213, Gueldener et al. (1996) Nucleic Acids Res. 24:2519-2524, and Smith et al. (1996) Methods Mol. Cell. Biol. 5:270-277.)

Once the host cell has been modified in this fashion the vector carrying the promoter—TAL construct may be transformed into the cell. Any method can be used to introduce the plasmid vector in the host cell. In fact, many methods for introducing plasmid vector into microorganisms such as bacteria and yeast are well known to those skilled in the art. For example, heat shock, lipofection, electroporation, conjugation, fusion of protoplasts, and biolistic delivery are common methods for introducing nucleic acid into bacteria and yeast cells [Ito et al., J. Bacterol. 153:163-168 (1983)]; and [Durrens et al., Curr. Genet. 18:7-12 (1990)]; and [Becker and Guarente, Methods in Enzymology 194:182-187 (1991)].

### Tyrosine Substrate—Fermentation Schemes

An important element in the present method for pHCA production is the supply of suitable amounts of tyrosine. Tyrosine may be supplied following growth and TAL expression in the TAL production host.

Alternatively the tyrosine may be supplied through co-fermentation with another organism that produces tyrosine. These tyrosine producers may be engineered for the over-production of tyrosine, or may be naturally tyrosine producers.

Examples of tyrosine over-producing strains that are suitable for the present method include, Microbacterium ammoniaphilum ATCC 10155, Corynebactrium lillium NRRL-B-2243, Brevibacterium divaricatum NRRL-B-2311, Arthrobacter citreus ATCC 11624, and Methylomonas SD-20. Other suitable tyrosine over-producers are known in the art, see for example Microbial production of L-tyrosine: A Review, T. K. Maiti et al, Hindustan Antibiotic Bulletin, vol 37, 51-65, 1995. Additionally an example of an Escherichia tyrosine over-producing strain that may be used is E. coli TY1, available from OmniGene Bioproducts, Inc. Cambridge, Mass. New strains that over-produce tyrosine may be identified, produced through mutation or genetic engineering, or otherwise obtained.

Where tyrosine is provided by another microorganism, several production schemes are possible. For example, the schemes may involve either two production hosts, (“two strain embodiment”) one producing tyrosine and the other engineered to express the TAL enzyme activity, or a single production host (“single strain embodiment”) where TAL expression and the production of tyrosine occur in the same host.

In an example of the two strain embodiment a tyrosine producer (host A) and a TAL production host (host B) are grown separately for the production of tyrosine and TAL enzyme, respectively. Subsequently, the tyrosine produced from fermentation A, which may be unpurified, partially purified, or fully purified, and cells from fermentation B are mixed in a reaction tank, allowing for the conversion of tyrosine to pHCA

Alternatively, strains A and B may be co-fermented in a single fermentation tank. First, the mixed culture is grown under conditions that favor the overproduction of tyrosine. Under these conditions, the expression of the TAL enzyme from strain B is suppressed by virtue of the tightly regulated promoter. Next, an inducer (e.g. arabinose) is added to the fermentation broth to induce expression of the TAL enzyme at neutral pH. Finally, the pH of the co-fermentation tank is raised to about 9.9 to allow pHCA bioconversion by TAL. The bioconversion is carried out until most tyrosine in the reaction tank is converted to pHCA.

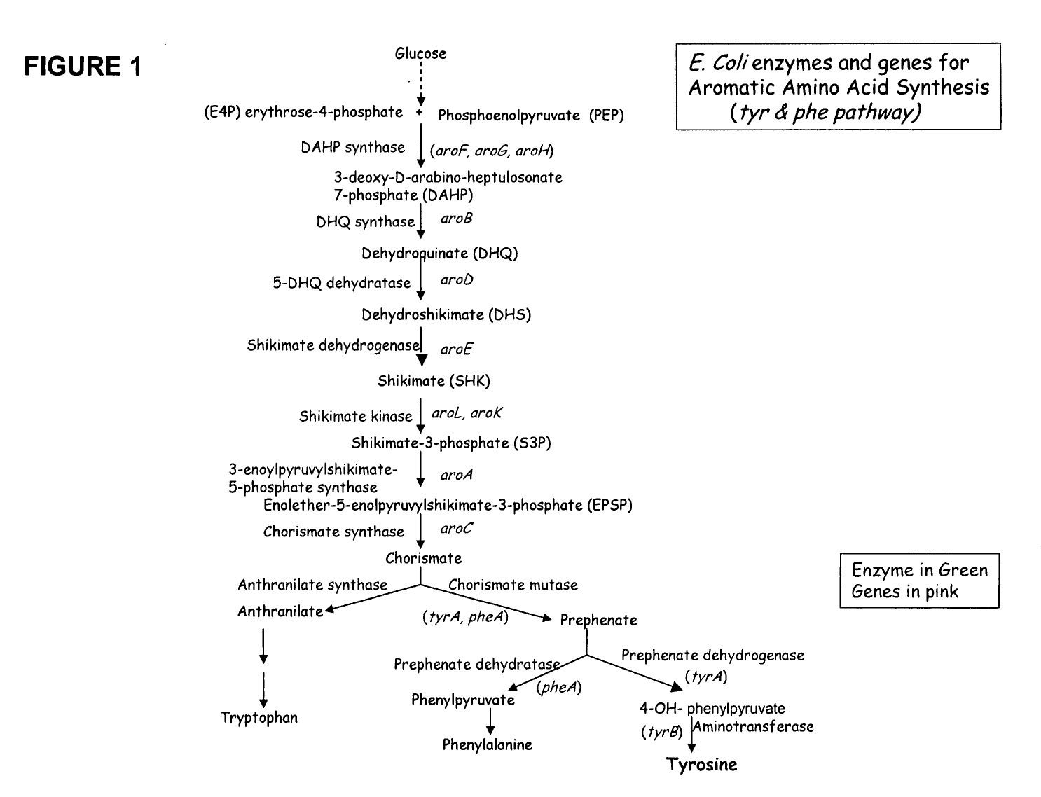

Alternatively the pHCA production strain may itself comprise a biosynthetic pathway for the synthesis of high levels of tyrosine (single strain embodiment). The pathway for tyrosine production will be based on the aromatic amino acid pathway, common in many microorganisms. The relevant elements of the aromatic amino acid pathway are illustrated in FIG. 1. Briefly, the pathway receives carbon ultimately from glucose and synthesis proceeds with the condensation of E4P and PEP to form DAHP, catalyzed by DAHP synthase, which is encoded by the aroFGH set of genes. The pathway proceeds though various intermediates catalyzed by the enzymes encoded to the genes aroB, aroD, aroE, aroL, arok, aroA and aroC, as shown in FIG. 1, to the point where chorismate is produced. Chorismate is a substrate for both anthranilate synthase (leading to trytophan synthesis) and chroismate mutase leading to the synthesis of first prephenate which itself may be acted on by prephenate dehydratase (encoded by pheA ) leading to phenylalanine synthesis, or prephenate deydrogenase (encoded by tyrA) leading first to the production of 4-OH-phenylpyruvate and then to tyrosine via catalysis by the tyrB encoded aminotransferase.

Given the elements of the pathway it will be apparent that the challenge in maximizing tyrosine production will be to control the loss of carbon to competing products (phenylalanine, tryptophan) and to optimize carbon flow toward the tyrosine product. Thus, up-regulation of the gene product of tyrA and elimination of gene product of pheA are indicated. Additionally, because wildtype DAHP synthases are known to be inhibited by the end products of the pathway (phenylalanine, tryptophan, tyrosine), and because this is the first enzyme in the pathway controlling carbon flow, it will be useful to obtain strains containing this mutant enzyme to decrease its regulation by end product.

Modulation of the genes in the aromatic amino acid pathway for the production of tyrosine may be accomplished using the tools available to the skilled molecular biologist (Sambrook, supra). For example gene down regulation may be accomplished by targeted gene disruption as described above. Alternatively, antisense technology may be employed for down regulating genes where the sequence of the target gene is known. To accomplish this, a nucleic acid segment from the desired gene is cloned and operably linked to a promoter such that the anti-sense strand of RNA will be transcribed. This construct is then introduced into the host cell and the antisense strand of RNA is produced. Antisense RNA inhibits gene expression by preventing the accumulation of mRNA which encodes the protein of interest. The person skilled in the art will know that special considerations are associated with the use of antisense technologies in order to reduce expression of particular genes. For example, the proper level of expression of antisense genes may require the use of different chimeric genes utilizing different regulatory elements known to the skilled artisan.

Although targeted gene disruption and antisense technology offer effective means of down regulating genes where the sequence is known, other less specific methodologies have been developed that are not sequence based. For example, cells may be exposed to a UV radiation and then screened for the desired phenotype. Mutagenesis with chemical agents is also effective for generating mutants and commonly used substances include chemicals that affect non-replicating DNA such as HNO₂ and NH₂OH, as well as agents that affect replicating DNA such as acridine dyes, notable for causing frameshift mutations. Specific methods for creating mutants using radiation or chemical agents are well documented in the art. See for example Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36, 227, (1992).

Another non-specific method of gene disruption is the use of transposable elements or transposons. Transposons are genetic elements that insert randomly in DNA but can be latter retrieved on the basis of sequence to determine where the insertion has occurred. Both in vivo and in vitro transposition methods are known. Both methods involve the use of a transposable element in combination with a transposase enzyme. When the transposable element or transposon, is contacted with a nucleic acid fragment in the presence of the transposase, the transposable element will randomly insert into the nucleic acid fragment. The technique is useful for random mutagenesis and for gene isolation, since the disrupted gene may be identified on the basis of the sequence of the transposable element. Kits for in vitro transposition are commercially available (see for example The Primer Island Transposition Kit, available from Perkin Elmer Applied Biosystems, Branchburg, N.J., based upon the yeast Ty1 element; The Genome Priming System, available from New England Biolabs, Beverly, Mass.; based upon the bacterial transposon Tn7; and the EZ::TN Transposon Insertion Systems, available from Epicentre Technologies, Madison, Wis., based upon the Tn5 bacterial transposable element.

Any of the above methods may be used by the skilled person to modify, up or down regulate, or disrupt various elements of the aromatic amino acid pathway to achieve tyrosine production in a TAL production host.

Fermentation Conditions

Once the desired TAL production host has been constructed it may be used for the high level production of pHCA. The optimal pH for TAL activity is about 8.0 to about 11.0, where a pH of about 9.5 to about 10.5 is preferred. Typically a pHCA production host expressing TAL is grown at physiological pH (about 6.5 to about 7.5) until it is ready to be contacted with a source of tyrosine. Once tyrosine is produced the pH is increased and the conversion of tyrosine to pHCA proceeds. This process is used irrespective of whether the tyrosine is supplied exogenously to the TAL host or as part of a whole cell system. Where the TAL host itself comprises a tyrosine pathway, the cells are grown at physiological pH until tyrosine accumulates and then the TAL is up-regulated by the addition of an inducer as the pH is increased to optimal levels.

Generally in this process it may be useful to control the levels of ammonia in the fermentation culture. For example, the presence of ammonia in the culture may have an inhibitory effect on the rates and yield of the TAL catalyzed reaction. Hence, removal of ammonia, preferably as it is formed in the fermentation, may enhance the rates and yields of this reaction. Removal of ammonia may be accomplished by means well known in the art. For example aeration or the addition of specific sorbents, such as the mineral clinoptilolite or ion exchange resins are typically suitable means.

For large scale commercial production it is expected that fermentations will take place in a fermentor. Any suitable fermentor may be used including a stirred tank fermentor, an airlift fermentor, a bubble fermentor, or any combination thereof. Materials and methods for the maintenance and growth of microbial cultures are well known to those in the art of microbiology or fermentation science (See for example, Bailey et al., Biochemical Engineering Fundamentals, second edition, McGraw Hill, N.Y., 1986).

Consideration must be given to appropriate growth medium, pH, temperature, and requirements for aerobic, microaerobic, or anaerobic conditions, depending on the specific requirements of the microorganism for the specific gene expression. The growth medium used is not critical, but it must support growth of the microorganism used and promote the enzymatic pathway necessary to produce the desired product. A conventional growth medium may be used, including, but not limited to complex media, containing organic nitrogen sources such as yeast extract or peptone and a fermentable carbon source; minimal media and defined media.

Suitable fermentable carbon sources include, but are not limited to monosaccharides, such as glucose or fructose; disaccharides, such as lactose or sucrose; oligosaccharides and polysaccharides, such as starch or cellulose; one-carbon substrates such as carbon dioxide, methanol, formaldehyde, formate, and carbon-containing amines and/or mixtures thereof. In addition to the appropriate carbon source, the growth medium must contain a suitable nitrogen source, such as an ammonia salt, yeast extract or peptone; minerals, salts, cofactors, buffers and other components, known to those skilled in the art (Bailey et al. supra).

Fermentations may proceed at an industrial scale under batch, fed-batch or continuous fermentation conditions. A classical batch fermentation is a closed system where the composition of the media is set at the beginning of the fermentation and not subject to alterations during the fermentation. Thus, at the beginning of the fermentation the medium is inoculated with the desired microorganism(s) and fermentation is permitted to occur adding nothing to the system. Typically, however, the concentration of the carbon source in a “batch” fermentation is limited and attempts are often made at controlling factors such as pH and oxygen concentration. In batch systems the metabolite and biomass compositions of the system change constantly up to the time the fermentation is stopped. Within batch cultures cells moderate through a static lag phase to a high growth log phase and finally to a stationary phase where growth rate is diminished or halted. If untreated, cells in the stationary phase will eventually die. Cells in the log phase generally are responsible for the bulk of production of end product or intermediate.

A variation on the standard batch system is the Fed-Batch system. Fed-Batch fermentation processes are also suitable in the present invention and comprise a typical batch system with the exception that the substrate is added in increments as the fermentation progresses. Fed-Batch systems are useful when catabolite repression is apt to inhibit metabolism of the cells and where it is desirable to have limited amounts of substrate in the medium. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch fermentations are common and well known in the art and examples may be found in [Brock, T. D.; Biotechnology: A Textbook of Industrial Microbiology, 2nd ed.; Sinauer Associates: Sunderland, Mass., 1989] or [Deshpande, M. V. Appl. Biochem. Biotechnol. 36:227, (1992)], herein incorporated by reference.

Commercial production of pHCA or tyrosine may also be accomplished with continuous fermentation. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in their log phase of growth.

Continuous fermentation allows for modulation of any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by the medium turbidity, is kept constant. Continuous systems strive to maintain steady state growth conditions and thus the cell loss due to the medium removal must be balanced against the cell growth rate in the fermentation. Methods of modulating nutrients and growth factors for continuous fermentation processes as well as techniques for maximizing the rate of product formation are well known in the art of industrial microbiology and a variety of methods are described by Brock, supra.

### Recovery of pHCA

Methods for the recovery of pHCA from a growth medium are available. One preferred method is taught in the co-pending and commonly owned U.S. patent application Ser. No. 10/824237, hereby incorporated by reference. Briefly the method involves first acidifying the fermentation broth containing either the pHCA to a pH or about 4.0 or below and then adding an extractant. Extractants useful for this purpose are water immiscible organic solvents and may includem but are not limited to, diisopentyl ether, n-propyl benzoate, 2-undecanone, dibenzyl ether, 2-tridecanone, 2-decanone, 1-pentanone 1-phenyl, methyl decanoate, 1-undecanol, diisobutyl DBE-IB and mixtures thereof. The pHCA or CA is dissolved in the extractant and removed from the medium. The pHCA or CA may then be recovered from the extractant by well known means such as distillation, adsorption by resins, or separation by molecular sieves. Alternatively, the pHCA may be recovered by acidification of the growth medium to a pH below 2.0, followed by crystallization.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions

### General Methods and Materials

Standard recombinant DNA and molecular cloning techniques used in the Examples are well known in the art and are described by Sambrook, J., Fritsch, E. F. and Maniatis, T., Molecular Cloning: A Laboratory Manual, Cold Spring Harbor Laboratory Press, Cold Spring Harbor, N.Y., 1989, by T. J. Silhavy, M. L. Bennan, and L. W. Enquist, Experiments with Gene Fusions, Cold Spring Harbor Laboratory, Cold Spring Harbor, N.Y., 1984, and by Ausubel, F. M. et al., Current Protocols in Molecular Biology, Greene Publishing Assoc. and Wiley-Interscience, N.Y., 1987.

Materials and methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or by Thomas D. Brock in Biotechnology: A Textbook of Industrial Microbiology, Second Edition, Sinauer Associates, Inc., Sunderland, Mass., 1989.

All reagents, restriction enzymes and materials used for the growth and maintenance of bacterial cells were obtained from Aldrich Chemicals (Milwaukee, Wis.), BD Diagnostic Systems (Sparks, Md.), Life Technologies (Rockville, Md.), DIFCO Laboratories (Detroit, Mich.). or Sigma Chemical Company (St. Louis, Mo.), unless otherwise specified.

### PCR Conditions

Unless specifically noted, all PCR reactions were carried out using the HotStarTaq Master Mix Kit (Qiagen, Valencia, Calif.). The Master Mix contained: 10 units/μl HotStarTaq DNA polymerase, PCR buffer with 3 mM MgCl₂, and 400 μM each dNTP. Reactions were carried out in 15 μl volumes in either 96-well plates or 0.2 mL eppendorf tubes. Five to ten pmoles of each primer were present, as well as either a small amount of cell material or 250 picograms of genomic DNA, prepared with the Puregene kit from Gentra Systems (Minneapolis, Minn.) as template. Samples were heated in a Mastercycler gradient thermocycler (Eppendorf, Westbury, N.Y.) to 95° C. for 15 min in order to activate the polymerase, then 30 cycles followed consisting of: 94° C. for 30 sec, 55° C. or 58° C. for 30 sec, and 72° C. for 1 min. Finally, the samples were kept at 72° C. for 10 min for final polymerization. The tubes or plate were then cooled to 4° C. until removed from the thermocycler.

### Strains and Vectors:

Construction of Recombinant Bacterial Strains: The recombinant bacterial strains used in the Examples were constructed using standard recombinant DNA and molecular cloning techniques that are well known in the art and are described in Maniatis, supra; Silhavy, T. J., Bennan, M. L. and Enquist, L. W., supra; and in Ausubel, F. M. et al., supra. A derivative of E. coli K12 strain BW25113 that contained a plasmid was obtained from Barry Warnner at Purdue University. The plasmid, which was temperature sensitive, was cured from the cells by growing at high temperature to provide the BW25113 strain. BW25113 is available as CGSC#7636 from the E. coli Stock Center at Yale University (New Haven, Conn.). Vector pBR322 was purchased from New England Biolab (Bevely, Mass.).

E. coli strain DPD5138 {[Lambda-rph-1 laclq rrnBT14 Δ(lacZ)WJ16 hsdR514 Δ(araBAD)AH33 Δ(rhaBAD)LD78] ΔthyA} was constructed as a background for the thymidylate synthase (thyA gene) selection marker. Since thymidylate synthase is involved in the do novo synthesis of dTTP from dUMP, a thyA knockout can grow on minimal media only with thymine supplementation. A thyA deletion in the BW25113 strain was constructed via lambda Red mediated recombination [Datsenko and Wanner (2000)Proc. Natl. Acad. Sci. USA 97:6640-6645; Ellis et al. (2001) Proc. Natl. Acad. Sci. USA 98:6742-6746] without the introduction of an antibiotic resistance selection marker as follows. The activity of thymidylate synthase requires tetrahydrofolate (THF) as a cofactor. THF is important for many other essential cellular reactions and is recycled via dihydrofolate reductase, which is a target for the inhibitor trimethoprim. An active thymidylate synthase coupled with an inactive dihydrofolate reductase results in accumulation of dihydrofolate that cannot be converted back to THF, which leads to cell death. Thus a thyA gene knockout can be positively selected by growing the cells on a minimal medium (e.g., M9, 0.4% glucose) in the presence of 0.8 mM thymine and 10 mg/mL trimethoprim.

A PCR fragment containing a truncated thyA gene was generated by fusion PCR using E. coli MG1655 genomic DNA as template. First, primers F1 (SEQ ID NO:16) and R2 (SEQ ID NO:17) were used to amplify a 400 bp region from the 3′ end of the upstream umpA gene to 100 bp downstream from the start codon of the thyA gene. The first 100 bp of thyA gene contains a transcriptional termination signal for umpA gene and is therefore essential to preserve in the knockout construct. Second, primers F2 (SEQ ID NO:18) and R1 (SEQ ID NO:19) were used to amplify a 360 bp fragment from the intergenic region between thyA and the downstream gene ppdA as well as part of the ppdA gene. The R2 primer also contains an overlapping region of 20 bp with the F2 primer, which enabled the fusion PCR reaction between PCR1 and PCR2 products, amplified with F1 and R1 primers, which yielded a 700 bp fragment. This fusion fragment was transformed into E. coli host BW25113{[Lambda-rph⁻¹ lacl^{q} rrnBT14 Δ(lacZ)WJ16 hsdR514 Δ(araBAD)AH33 Δ(rhaBAD)LD78]} (transformed with pKD46) by electroporation, and plated on M9 media, 0.4% glucose, 0.8 mM thymine and 10 mg/mL trimethoprim. Plasmid pKD46 contains the lambda Red recombinase gene which was induced by arabinose. The plate was incubated at 37° C. for 2 days, then 16 colonies were picked and re-streaked on M9 glucose and M9 glucose plus thymine plates for analysis and colony purification. A number of clones grew on M9 glucose plus thymine, but not on M9 glucose plates. These clones were incubated at 42° C. to eliminate the temperature sensitive plasmid pKD46. The new strain generated, DPD5138 {[Lambda-rph⁻¹ lacl^{q} rrnBT14 Δ(lacZ)WJ16 hsdR514 Δ(araBAD)AH33 Δ(rhaBAD)LD78] ΔthyA}, therefore is thymine auxotroph. The thyA deletion was confirmed by PCR of the chromosomal DNA of DPD5138.

### Growth Conditions and Materials

Typically, bacteria were incubated overnight at 37° C. in LB medium in 15-ml snap cap tubes or 125 ml flasks.

### Protein Assay

Protein concentrations were determined by Bradford protein assay, using a BioRad kit (BioRad Laboratories, Hercules, Calif.) for protein determination. A protein standard curve was generated using five protein standards ranging from 100 to 800 μg/mL bovine serum albumin (BSA) solutions. The microtiter plate assay format was used which required the addition of 10 μL of sample to 160 μL of the BioRad reagent.

### Enzyme Activity Assay

The PAL or TAL activity of the purified enzymes was measured using a spectrophotometer according to Abell et al. (Methods Enzymol. 142:242-248 (1987)). The spectrophotometric assay for PAL determination was initiated by the addition of the enzyme to a solution containing 1.0 mM L-phenylalanine and 50 mM Tris-HCl (pH 8.5). The reaction was then followed by monitoring the absorbance of the product, CA, at 290 nm using a molar extinction coefficient of 9000 cm⁻¹. The assay was run over a 5 min period using an amount of enzyme that produced absorbance changes in the range of 0.0075 to 0.018/min. One unit of activity indicated deamination of 1.0 micro-mol of phenylalanine to CA per minute. The TAL activity was similarly measured using tyrosine in the reaction solution. The absorbance of the pHCA produced was followed at 315 nm and the activity was determined using an extinction coefficient of 16,800 M⁻¹ cm⁻¹ for pHCA. One unit of activity indicated deamination of 1.0 micro-mol of tyrosine to pHCA per minute.

### Gel Electrophoresis

A 4-12% gradient BIS-TRIS gel (Invitrogen, Carlsbad, Calif.) was loaded with 4.0 μg of protein per lane, run at 200 v, and stained with Simply Blue SafeStain (Invitrogen). The High Molecular Weight (HMW) marker was used as the molecular weight standards (Amersham, GE Healthcare, Piscataway, N.J.). The gel image was analyzed using Kodak 1D Image Analysis Software (Kodak).

### HPLC Analysis

The mixture of phenylalanine, tyrosine, cinnamic acid and para-hydroxycinnamic acid (pHCA) can be analyzed using the following HPLC method:

An Agilent 1100 System (Agilent technologies, Palo Alto, Calif.) with a photodiode array detector and a Zorbax SB-C18 column (3.5 μm, 4.6×150 mm-rapid resolution) is used and separation is achieved by a gradient combining two solvents: Solvent A, 0.1% trifluoroacetic acid in water; Solvent B, 0.1% trifluoroacetic acid in acetonitrile. The method requires a column flow rate of 1.0 mL/min, with a run time of 15 minutes and a post-run time of 5 minutes. The solvent gradient used is that given in Table 1 below. The pump runs within pressure limits defined as a minimum of 20 bar and a maximum of 400 bar. Solutions are filtered through an 0.45-micron nylon filter before dilution in HPLC-grade water and transfer into HPLC vial and injection. The sample spectrum is scanned from 100 nm to 380 nm, and the signal for phenylalanine at (215 or 220 nm), tyrosine at (278 nm), Cinnamic acid at (278 nm) and pHCA at (312 nm) are measured. Table 2 shows the elution times for the compounds of interest using this method.

TABLE 1
Solvent Gradient Used for HPLC

| Time (min) | Solvent A | Solvent B |
|---|---|---|
| 0 | 95% | 5% |
| 8 | 20% | 80% |
| 10 | 20% | 80% |
| 15 | 95% | 5% |

TABLE 2
Elution times for various compounds of interest

| Compound | Elution time -min (+/−0.1) |
|---|---|
| phenylalanine | 4.4 |
| tyrosine | 3.5 |
| Cinnamic acid | 7.2 |
| pHCA | 5.3 |

The meaning of abbreviations is as follows: “sec” means second(s), “min” means minute(s), “h” means hour(s), “d” means day(s), “μL” means microliter, “mL” means milliliters, “L” means liters, “mm” means millimeters, “nm” means nanometers, “mM” means millimolar, “M” means molar, “mmol” means millimole(s), “μmole” mean micromole”, “g” means gram, “μg” means microgram and “ng” means nanogram, “U” means units, and “mU” means milliunits.

### CA is cinnamic acid.

### Example 1

### TAL Enzyme Activity Production Using Tac Promoter Expression of RgTAL

In U.S. Pat. No. 6,521,748, which is herein incorporated by reference, the RgTAL (therein called Rhodosporidium toruloides PAL) coding region was cloned behind the tac promoter in the KK223-3 vector (Amersham Pharmacia). The resulting pKK223-PAL/TAL vector was transformed into the E. coli TOP10 strain (Invitrogen) and called strain TOP10(pKK223.TAL). Expression of RgTAL enzyme activity was analyzed in this strain. Cells were initially grown overnight, at 37° C. on 50 mL LB media with 100 mg/L ampicillin in a baffled 250 mL flask. Before harvesting the non-induced cells, a 5.0 mL aliquot was transferred into fresh medium and grown to about 0.9 (OD₆₀₀). IPTG (isopropyl-β-thiogalactoside) was then added to a final concentration of 0.2 mg/mL to induce the enzyme and the cells were further grown for 18 h.

Samples of induced and uninduced TOP10(pKK223.TAL) cultures were analyzed for TAL activity as described in Example 3 below. Even in the absence of the inducer IPTG these cells had about 30 U/g of TAL activity. Following addition of 1.0 mM IPTG, only a slight increase in the TAL activity was observed to 35 U/g after 18 h of induction. Thus there was a significant lack of tight control of the TAL enzyme expression from the pKK223-PAL/TAL vector. It is thought that the presence of the TAL activity in the “uninduced cells” resulted in conversion of the intracellular tyrosine to pHCA, which is toxic and may cause cell damage. Such cell damage may result in the low “induced” expression and TAL activity after IPTG addition. Thus the amount of accumulated TAL activity in cells with expression of RgTAL from the induced tac promoter is lower than desired for use in pHCA production.

The identification of a tightly regulated system for TAL expression was therefore important for: 1) prevention of cell damage resulting from the presence of low levels of TAL in “uninduced” cells; and 2) increasing the levels of TAL activity to be used in pHCA production. In addition, IPTG is an expensive ingredient and its replacement with a lower cost inducer, for large scale production, would be desirable. A significantly lower cost inducer is arabinose, making it a much more attractive inducer for commercial applications. Therefore RgTAL expression from the inducible araB promoter was tested.

### Example 2

### Construction of Arabinose Inducible Expression Vector for RqTAL Enzyme

The purpose of this example was to clone the gene encoding the TAL enzyme from Rhodotorula glutinis into a medium copy number expression vector for the high level inducible expression of Rhodotorula glutinis TAL (abbreviated as RgTAL).

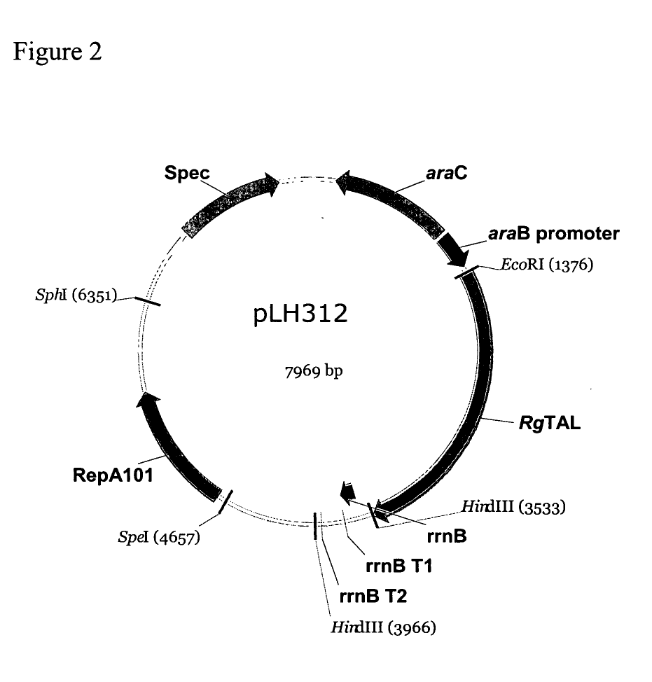

pLH320 is a medium copy number expression vector used for the high level inducible expression of the RgTAL coding region. pLH320 was constructed starting with pCL1920, a low copy number plasmid with the SC101 origin of replication and spectinomycin resistance marker, obtained from Netherlands Culture Collection of Bacteria (NCCB). The E. coli K12 araC gene encoding the transcriptional activator for the araB promoter, and the araB promoter were cloned into pCL1920. The araC-araB region was PCR amplified as a cassette from E. coli strain FM5 (ATCC#53911) genomic DNA using primers of SEQ ID NOs:20 and 21. The resulting PCR fragment was digested with AosI and HindIII, and ligated to pCL1920 digested with AosI and HindIII. Plasmid DNA of colonies resulting from transformation was isolated and assayed by restriction digestion and sequencing to confirm the desired construction, called pCL1920ara. An RgTAL coding region DNA fragment was excised from plasmid pKK223-PAL (described in U.S. Pat. No. 6,521,748) by EcoRI, HindIII digestion and ligated into EcoRI, HindIII digested pCL1920ara to give pCL1920ara.mcs.PAL. The transcription termination sequences rrnBT1 and rrnBT2 were PCR amplified from plasmid pTrc99A (Pharmacia Biotech, Amersham, GE Healthcare, Piscataway, N.J.) using primers of SEQ ID NOs:22 and 23, and digested with HindIII, which cuts at both 5′ and 3′ ends of the PCR product. The rrnBT1&2 fragment was cloned into the HindIII site of pCL1920ara.mcs.PAL, 3′ to the araB promoter to yield plasmid pLH312 (FIG. 2). This plasmid was converted to a medium copy number plasmid in two steps. First, a linker was inserted by site-directed mutagenesis to replace the HindIII site in pLH312 between the rrnBT2 transcription terminator and the SC101 origin of replication. This linker contains Kpnl, Xbal and Spel sites for the subsequent cloning of the colE1 replication origin. Two complementary oligonucleotides encoding for the linker sequence (SEQ ID NOs: 24 and 25 were used to perform a site-directed mutagenesis reaction with pLH312 as template using the Quick Change Site-Directed Mutagenesis Kit (Stratagene, San Diego, Calif.). Upon sequencing confirmation, the new plasmid was named pLH319. The colE1 replication origin and rop (encodes a replication origin protein) gene locus of pBR322 were PCR amplified using primers of SEQ ID NOs:26 and 27. The resulting 1.8 kb PCR fragment was digested with Sphl and Spel, and ligated with pLH319 which was digested with Sphl and Spel. This yielded plasmid pLH320, which contains the colE1 replication origin in place of SC101 origin. pLH320 was transformed into E. coli K12 strain BW25113 [Lambda-rph-1 laclq rrnBT14 Δ(lacZ)WJ16 hsdR514 Δ(araBAD)AH33 Δ(rhaBAD)LD78) to generate strain DPD5124. A derivative of BW25113 that contained a plasmid was obtained from Professor Barry Warnner at Purdue University. The plasmid, which was temperature sensitive, was cured from the cells by growing at high temperature to provide the BW25113 strain. BW25113 is available as CGSC#7636 from the E. coli Stock Center at Yale University (New Haven, Conn.).

### Example 3

### Improvement in Enzyme Production Using araB Promoter Expression of RgTAL Enzyme

The purpose of this example was to analyze the TAL activity in strain DPD5124, with RgTAL expressed from the induced araB promoter.

Fresh colonies of strain DPD5124 were separately inoculated into 5.0 ml of LB medium supplemented with 50 μg/ml spectinomycin and incubated overnight at 37° C. with shaking at 250 rpm. Each culture was then diluted to an optical density (OD₆₀₀) of 0.02 in the LB medium with 50 μg/ml spectinomycin and grown to an OD₆₀₀ of 0.4, at which time they were induced with 0.2% to 0.00002% of L-arabinose. The induced cultures were incubated for 20 hours at 37° C. with shaking at 250 rpm after which the cells were pelleted by centrifugation at 2,300×g, 4° C. for 30 minutes in a Beckman GS-6R (Fullerton, Calif.) centrifuge. The pellets were re-suspended in 2.0 ml of ice cold 50 mM Tris-HCl, pH 8.5 containing the Protease inhibitor cocktail (Roche, Palo Alto, Calif.), transferred to ice cold 15 ml sterile conical tubes and sonicated in a Fisher Sonic Model 300 Dismembrator (Pittsburgh, Pa.) at 50% power with repeating four cycles of 30 seconds sonication followed by 60 seconds rest in between each cycle. The samples were kept in an ice bath during the entire procedure. The sonicated samples were centrifuged at 15,000×g for 30 minutes at 4° C. to separate the crude cell extracts into soluble (supernatant) and insoluble (pellet) protein fractions. The resulting pellet was re-suspended in 1.0 ml of ice cold 50 mM Tris, pH 8.5 containing the Protease inhibitor cocktail. The protein concentration of the soluble samples was determined using the Bradford protein assay (Bio-Rad, Hercules, Calif.). Samples were then diluted with 50 mM Tris-HCl, pH 8.5 until the optical density (OD₅₉₅ nm) was in the linear range of a bovine serum albumin (BSA) standard curve (0.125-1.0 mg/ml). The TAL activity in the soluble fraction was determined by measuring production of pHCA. The assay was performed in a 1.5 ml capacity UV grade disposable cuvette (VWR) at 35° C. using a Perkin-Elmer Lamba20 spectrophotometer (Wellesley, Mass.). The reaction (1.0 ml volume) which contained the soluble protein sample, 100 mM CAPS pH10 buffer and 10 mM tyrosine was monitored for 3 minutes at λ315 nm. The TAL activity (U/g) was calculated as described below: Total TAL activity (μM/min)=Δ315 nm/min×1,000,000 (μM/M) divided by pHCA extinction coefficient (M⁻¹ cm⁻¹)=Δ315 nm/min ×1,000,000 (□M/M) divided by 16,800 (M⁻¹ cm⁻¹). TAL specific activity (U/g)=total TAL activity (μM/min) divided by the amount of protein used in the assay.

The amounts of TAL activities in the soluble fractions following induction with each different arabinose concentration are given in Table 3. High levels of TAL activity were present after induction with all concentrations of arabinose, though 0.0002% arabinose was not as effective as higher concentrations. No TAL activity was present in uninduced controls.

TABLE 3
TAL activity obtained at 0.2% to 0.0002% arabinose inducer
concentrations.

| Arabinose | TAL activity (U/g |
|---|---|
| concentration (%) | protein) |
| 0.2 | 207 |
| 0.02 | 241 |
| 0.002 | 216 |
| 0.0002 | 99 |

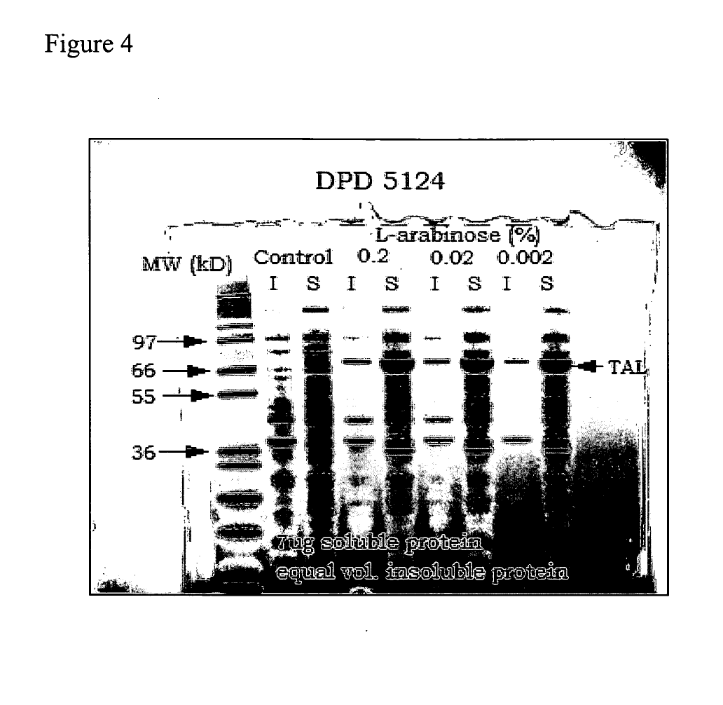

The level of RgTAL protein accumulation in the DPD5124 strain was assayed as follows: 7.0 μg of each protein sample (soluble and insoluble fractions) was diluted 1:1 with 2× Laemmli protein sample buffer (Bio-Rad, Hercules, Calif.), denatured at 100° C. for 10 min, then loaded onto a 4-12% gradient Bis-Tris gel (Invitrogen) and run at 200 V for approximately one hour. Each gel was then rinsed with double distilled water for 5 minutes and stained with Simply Blue SafeStain (Invitrogen) for 45 minutes. The stained gels were de-stained by repeated washes with Millipore water while shaking gently until the non-protein background became transparent. The de-staining was typically performed overnight at room temperature with gentle orbital shaking. The images of the stained gels were captured using a Kodak Gel Logic 100 imaging system. As seen on the gel in FIG. 4, a large amount of RgTAL protein was present in the soluble fractions from the 0.2%, 0.02%, and 0.002% arabinose induction samples. The 0.0002% arabinose sample was not included. No RgTAL protein was seen in the uninduced control sample.

### Example 4

### Construction of RqTAL Expression Strain with Non-Antibiotic Selection Marker

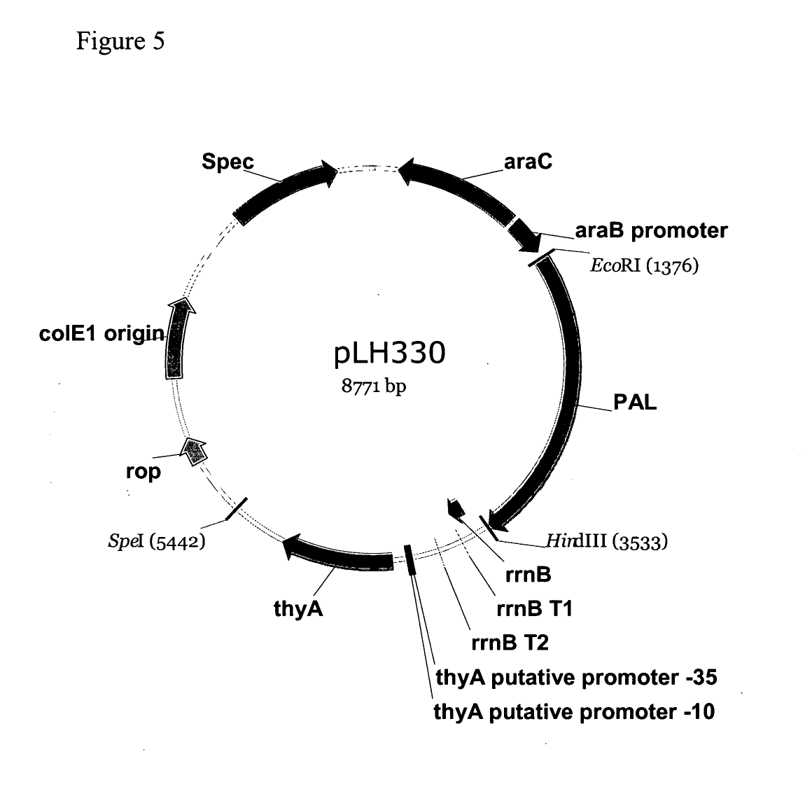

The purpose of this example was to engineer a TAL production strain that does not require any antibiotics for maintaining the TAL expression plasmid. To do this, a non-antibiotic selection marker, thyA, which encodes for thymidylate synthase, was inserted into the plasmid pLH320. The thyA locus of E. coli MG1655 (ATCC #47076), including the thyA coding region, the intrinsic thyA promoter located within the upstream gene umpA, and a transcriptional terminator in the downstream gene ppdA, was amplified by PCR using a 5′ primer (SEQ ID NO:28) and a 3′ primer (SEQ ID NO:29). The PCR primers contained Xbal and Spel sites, and the PCR product (1.5 kb) digested with these enzymes was ligated into the pLH320 plasmid at the Spel and Xbal sites to generate pLH330 (FIG. 5). The pLH330 plasmid was transformed into E. coli host DPD5138 {[Lambda-rph-1 laclq rrnBT14 Δ(lacZ)WJ16 hsdR514 Δ(araBAD)AH33 Δ(rhaBAD)LD78] ΔthyA}, the construction of which is described in General Methods, to produce strain DPD5142. pLH330 was able to complement the thymine auxotrophy of the host strain DPD5138, indicating that the thyA gene was functionally expressed on the plasmid pLH330 in the DPD5142 strain.

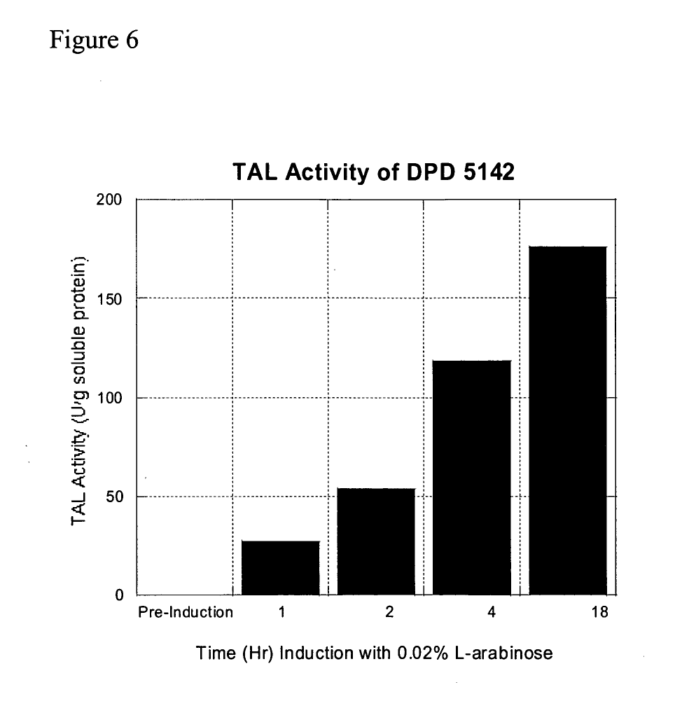

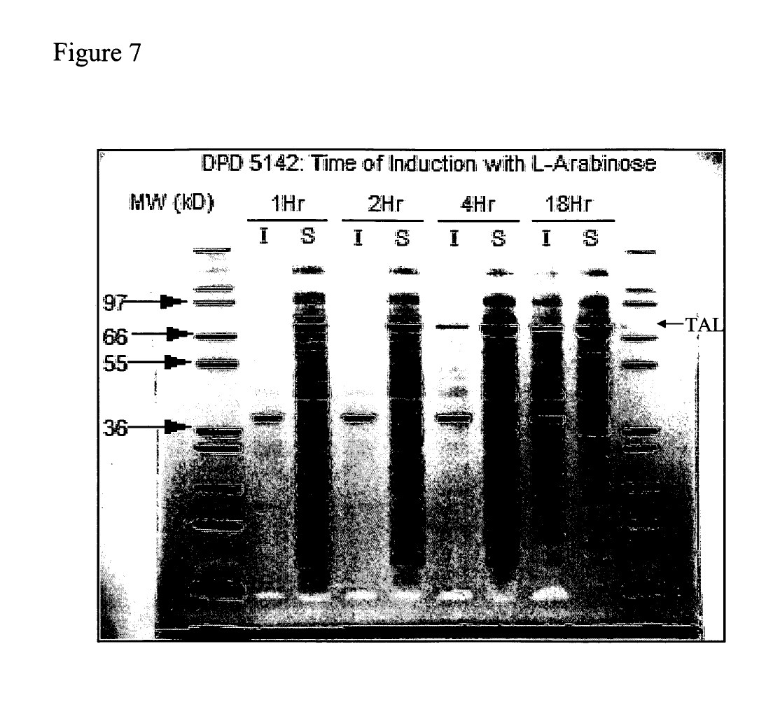

The TAL activity of DPD5142 was measured as described in the previous example. RgTAL enzyme expression was induced by 0.02% arabinose for 1, 2, 4, and 18 h, and the activity (shown in FIG. 6) and expression (shown in FIG. 7) were studied. DPD5142 and the previous strain DPD5124 had similar levels of RgTAL protein expression and enzyme activity. In addition, the TAL activity of DPD5142 was stable after >50 generations of continuous culturing in M9 minimal growth medium containing 0.2% glucose, in the absence of antibiotics. The DPD5142 strain is therefore suitable for large scale antibiotics-free fermentation.

### Example 5

### Optimization of Induction of RgTAL Expression in DPD5124 Using Arabinose

All fermentations were done in a 10 liter Braun BiostatC fermentor with an initial volume post inoculation of 8 liters of medium containing: yeast extract (2.0 g/L), CaCl₂.2H₂O (0.8 g/L), citric acid.H₂O (1.9 g/L), FeSO₄.7H₂O (0.2 g/L), MgSO₄.7H₂O (1.1 g/L), MnSO₄.H₂O (0.03 g/L), NaCl (0.01 g/L), ZnSO₄.7H₂O (1.0 mg/L), H₃BO₃ (0.1 mg/L), CuSO₄.5H₂O (0.1 mg/L), NaMoO₄.2H₂O (0.1 mg/L), phosphoric acid, 85% (2.9 mL/L), sulfuric acid, 98% (0.5 mL/L), KOH, 50% (0.275 mL/L), and antifoam (0.5 mL/L). Just prior to inoculation, glucose and spectinomycin were added to final concentrations of 5 g/L and 50 mg/L, respectively. The DPD5124 inoculum was grown in a 2 L shake flask containing 500 mL of the following medium: KH2PO4 (2.0 g/L), K₂HPO₄ (13.0 g/L), (NH₄)₂PO₄ (4 g/L), MgSO₄.7H₂O (1.0 g/L), yeast extract (2.0 g/L), ferric ammonia citrate (0.1 g/L), glucose (5.0 g/L) and spectinomycin (50 mg/L), with pH adjusted to 6.8; the shake flask was incubated at 36° C. at 300 rpm to an OD₅₅₀ of 3 and the entire contents used to inoculate the fermenter. The fermenter was controlled at 36° C., pH 6.8[with NH₄OH, 40% (w/v)], airflow of 4 SLPM, pressure of 0.5 barg, and dissolved oxygen tension of 25%. A solution of glucose (50% (w/w)) was fed to the fermenter via an exponential ramp of 0.2/hr.

To induce RgTAL expression, arabinose was added to the fermenter in separate fermentation runs at the OD₅₅₀ and concentrations shown in Table 4. Each fermentation was continued for 15 hours and a final sample drawn for TAL analysis by specific activity (U/g dcw) or total activity in the tank (U). Results of the assays are shown in Table 4.

TABLE 4
TAL activity following induction with different amounts of
arabinose at varying cell concentrations.

| | induction | [arabinose] | TAL activity | Total TAL |
|---|---|---|---|---|
| Run # | OD₅₅₀ | (g/L) | (U/g dcw) | (U) |
| TAL011 | — | 0 | 87 | 2.0 × 10⁴ |
| TAL026 | 35 | 0.03 | 113 | 3.0 × 10⁴ |
| TAL002 | 35 | 0.3 | 163 | 3.4 × 10⁴ |
| TAL003 | 35 | 0.6 | 167 | 3.5 × 10⁴ |
| TAL027 | 70 | 0.3 | 169 | 5.2 × 10⁴ |
| TAL004 | 70 | 0.6 | 148 | 3.8 × 10⁴ |

In some fermentations, the temperature was changed at the time of induction, as given in Table 5. In these runs, the same concentration of arabinose (0.3 g/L) was used in each experiment. The results are shown in Table 5.

TABLE 5
TAL activity with induction at different cell concentrations and
temperatures.

| | induction | Temperature | TAL activity | Total TAL |
|---|---|---|---|---|
| Run # | OD₅₅₀ | at induction | (U/g dcw) | (U) |
| TAL014 | 35 | 36 | 134 | 2.8 × 10⁴ |
| TAL015 | 35 | 30 | 213 | 5.0 × 10⁴ |
| TAL016 | 35 | 24 | 196 | 4.3 × 10⁴ |
| TAL027 | 70 | 36 | 169 | 5.2 × 10⁴ |
| TAL030 | 70 | 30 | 134 | 5.8 × 10⁴ |

1. An E. coli K12 bacterial production host comprising at least one genetic construct encoding a polypeptide having tyrosine ammonia lyase activity, operably linked to a tightly regulated inducible promoter wherein the inducible promoter is selected from the group consisting of arabinose inducible promoter (araB), and rhamnose inducible promoter (rhaB).

2. (canceled)

3. (canceled)

4. A method for the production of tyrosine ammonia lyase comprising:
a) providing an E. coli K12 bacterial production host comprising at least one genetic construct encoding a polypeptide having tyrosine ammonia lyase activity operably linked to a tightly regulated inducible promoter selected from the group consisting of arabinose inducible promoter (araB) and rhamnose inducible promoter (rhaB);
b) growing the E. coli K12 bacterial production host of step a) in a growth medium; and
c) inducing the inducible promoter of step a) whereby tyrosine ammonia lyase is produced.

5. The method according to claim 4 whereby the tyrosine ammonia lyase produced at step c) comprises at least about 10% of soluble cellular proteins.

6. The method according to claim 4 whereby the tyrosine ammonia lyase produced at step c) comprises at least about 20% of soluble cellular proteins.

7. The method according to claim 4 whereby the tyrosine ammonia lyase produced at step c) comprises at least about 50% of soluble cellular proteins.

8. The method according to claim 4 whereby the tyrosine ammonia lyase produced at step c) comprises at least about 70% of soluble cellular proteins.

9. A method for the production of p-hydroxycinnamic acid comprising:
a) providing a source of tyrosine;
b) growing an E. coli K12 bacterial host comprising at least one genetic construct encoding a polypeptide having tyrosine ammonia lyase activity operably linked to a tightly regulated inducible promoter;
c) inducing the inducible promoter of b) whereby tyrosine ammonia lyase is produced in the E. coli K12 bacterial host;
d) combining the tyrosine of step (a) with the E. coli K12 bacterial host of step c) whereby p-hydroxycinnamic acid is produced; and
e) optionally recovering the p-hydroxycinnamic acid.

10. The method of claim 9 wherein the source of tyrosine is a bacterial host that is overproducing for tyrosine.

11. A method for the production of p-hydroxycinnamic acid comprising:
a) providing an E. coli K12 bacterial host comprising:
i) at least one genetic construct encoding a polypeptide having tyrosine ammonia lyase activity operably linked to a tightly regulated inducible promoter; and
ii) an endogenous source of tyrosine;
b) growing the E. coli K12 bacterial host of (a) under conditions whereby tyrosine is produced in the absence of tyrosine ammonia lyase activity;
c) inducing the inducible promoter of step (a)(i) where in the at least one genetic construct expresses the polypeptide having tyrosine ammonia lyase activity whereby p-hydroxycinnamic acid is produced; and
d) optionally recovering the p-hydroxycinnamic acid of step (c).

12. A method for the production of p-hydroxycinnamic acid comprising:
a) providing an E. coli K12 bacterial host comprising at least one genetic construct encoding a polypeptide having tyrosine ammonia lyase activity operably linked to a tightly regulated inducible promoter;
b) providing a bacterial production host that is overproducing for tyrosine;
c) cofermenting the E. coli K12 bacterial host of step (a) and the bacterial tyrosine overproducing host of step (b) in a single fermentation phase;
d) Inducing the cofermented E. coli K12 bacterial host of step (c) whereby tyrosine ammonia lyase is expressed and p-hydroxycinnamic acid is produced; and
d) optionally recovering the p-hydroxycinnamic acid of step (d).

13. A method for the production of p-hydroxycinnamic acid comprising:
a) growing an E. coli K12 bacterial host in a growth medium comprising at least one genetic construct encoding a polypeptide having tyrosine ammonia lyase activity operably linked to a tightly regulated inducible promoter;
b) providing tyrosine in the growth medium of (a);
c) inducing the inducible promoter whereby tyrosine ammonia lyase is expressed and p-hydroxycinnamic acid is produced; and
d) optionally recovering the p-hydroxycinnamic acid of step (c).

14. A method according to any of claims 9, 11, 12 or 13 wherein the polypeptide having tyrosine ammonia lyase activity has an amino acid sequence selected from the group consisting of SEQ ID NO: 1-12.

15. A method according to any of claims 9, 11, 12 or 13 wherein the tightly regulated inducible promoter is selected from the group consisting of araB, and rhaB.

16. An E. coli K12 bacterial production host comprising:
a) at least one genetic construct encoding a polypeptide having tyrosine ammonia lyase activity operably linked to a tightly regulated inducible promoter; and
b) an endogenous source of tyrosine.
